(19) United States
(12) Patent Application Publication
(10) Pub. No.: US 2012/0328696 A1
(43) Pub. Date: Dec. 27, 2012

(54) ANTI-PARKINSONIAN COMPOUND ACETYLSALICYLIC ACID MALTOL ESTER

(75) Inventor: Hyoung-Chun Kim, Chunchon (KR)

(73) Assignees: Hutecs Korea Pharmaceutical Co., Ltd., Seoul (KR); Kangwon National University, Gangwon-do (KR)

(21) Appl. No.: 13/599,598

(22) Filed: Aug. 30, 2012

Related U.S. Application Data
(62) Division of application No. 12/540,953, filed on Aug. 13, 2009.
(60) Provisional application No. 61/090,326, filed on Aug. 20, 2008.

(51) Int. Cl. A61K 31/621; A61K 9/48; A61P 25/16

(52) U.S. Cl. 424/451; 514/159

(57) ABSTRACT

The present application describes a composition comprising a neuroprotective effective amount of an antioxidant acetylsalicylic acid maltol ester (AME).

### BACKGROUND OF THE INVENTION

1. Field of the Invention

The invention relates to a neuroprotective compound. The invention further relates to a compound used to treat a variety of neurological conditions, including Parkinson's disease or the symptoms of Parkinson's disease, and learning and memory impairment in Alzheimer's disease.

2. General Background and State of the Art:

Methamphetamine (MA) is a widely potent and highly addictive stimulant that can lead to neurotoxicity in rodents, nonhuman primates and humans. It has been reported that MA administration induces increase in reactive oxygen species (ROS) formation, protein oxidation, and lipid peroxidation (Jayanthi et al., 1998; Gluck et al., 2001), which may involve in MA-mediated dopaminergic toxicity (Cadet et al., 1994; Giovanni et al., 1995; Yamamoto et al., 1998; Cadet and Brannock, 1998; Tsao et al., 1998; Fumagalli et al., 1999; La Voie and Hastings, 1999). Thus, the dopaminergic damage induced by MA can be one of the important models of the Parkinson's disease (Sonsalla et al., 1996; Davidson et al., 2001; Guilarte et al., 2001; Kita et al., 2003). Administration of antioxidants such as ascorbate and vitamin E attenuated MA-induced neurotoxicity (Wagner et al., 1985; De Vito and Wagner, 1989). In addition, transgenic (Tg) mice with CuZn-superoxide dismutase (SOD1) have been resistant to MA toxicity (Przedborski et al., 1992; Cadet at al., 1994). MA administration can lead to significant augmentation of hydrogen peroxide (H₂O₂), an important determinant in neural injury (Cubells et al., 1994; Yokoyama et al., 1997; Cadet and Brannock, 1998; Taylor et al., 2005).

Enzymatic antioxidants including catalase and glutathione peroxidase (EC 1.11.1.9, GPx), provide a first line of defense against H₂O₂. Since catalase is mainly expressed in peroxisomes and its activity is low in the brain (Halliwell, 1992), GPx is considered as a major H₂O₂ scavenger in brain. It catalyzes the degradation of H₂O₂ and hydroperoxides into water and alcohols, respectively, through the glutathione (GSH) redox cycle. Out of GPx isoenzymes, GPx-1 is a cytosolic selenium (Se)-dependent enzyme, which is ubiquitously expressed and plays a crucial role in removing peroxides in the brain. Without concommitent increase in the level and activity of GPx-1, H₂O₂ can accumulate, which can be metabolized into noxious hydroxyl radicals through Fenton reaction (Amstad et al., 1991; Teixeira and Meneghini, 1995).

The trace element Se is a constituent of the GPx1 (Rotruck et al., 1973; Huang et al., 1994). Se binds to the active site as a selenocysteine, a redox center in catalysis. Accumulating evidences indicate a positive correlation between GPx1 activity, dietary Se levels and resistance to oxidative stress (Castano et al., 1993; Huang et al., 1994; Jimenez-Jimenez et al., 1995). We reported that prolonged Se-deficiency potentiated MA-induced oxidative stress in the nigrostriatal system, while Se-repletion significantly prevents this toxicity, suggesting that the neuroprotective action of Se is mediated by a GSH-responsible antioxidant mechanism (Kim et al., 1999; Kim et al., 2000c).

Early studies suggested that microglial activation represents an early step in methamphetamine-induced neurotoxicity (Thomas et al., 2004; 2005). Once activated microglia can produce proinflammatory cytokines such as IL-6 and TNFα. They can initiate and promote inflammation in brain tissue (Kreutzberg, 1996; Stollg and Jander, 1999; Streit et al., 1999; Lavoie et al., 2004).

Brain-derived growth factor (BDNF) and glial cell line-derived neurotrophic factor (GDNF) belong to two different families of neurotrophic factors (NTFs) (Lapchak et al., 1996; Lewin and Barde, 1996; Shen et al., 1997). It has been proposed that BDNF plays a critical role in the survival and differentiation of midbrain dopaminergic neurons in vitro and in vivo (Hyman et al., 1991; Spina et al., 1992). Behavioral studies have demonstrated that local administration of BDNF or other neurotrophic factors can augment nigrostriatal DAergic functioning and locomotor behavior (Martin-Iverson et al., 1994; Horger et al., 1998; Pierce et al., 1999). GDNF has also been implicated in the survival and function of DA neurons and it can protect against dopaminergic degeneration (Bowenkamp et al., 1995; Tomac et al., 1995).

Recent findings suggested that protein kinase C(PKC) plays an important role in the dopaminergic neurotoxicity (McMillian et al., 1997; Fiebich et al., 1998; Li et al., 2006). It has been found that selective PKC inhibitors from different chemical classes (chelerythrine, calphostin C, and Ro31-8220) block amphetamine-induced DA release in rat striatum (Kantor and Gnegy, 1989; Brownan et al., 1998). Another PKC inhibitor, NPC 15437, also prevented the MA-induced oxidative effect in rat striatal synaptosomes (Pubill et al., 2005). Consistently, inactivation of PKC attenuated oxidative stress-mediated apoptotic dopaminergic neuronal death (Kaul et al., 2005). Similarly, amphetamine-stimulated dopamine efflux was enhanced with PKCβII activation (Johnson et al., 2005). Several groups showed that PKC-mediated phosphorylation of dopamine transporter (DAT) may affect its function after MA treatment (Kim et al., 2000; Sandoval et al., 2001). However, role of PKC on the MA-induced dopaminergic neurotoxicity remains elusive.

Ghanooni et al. (2006) showed that there was correlation between PKC isoforms and protein 53 (p53). p53 is a tumor suppressor gene whose activation has been associated with apoptosis. Recent studies have demonstrated that degeneration of dopaminergic cells induced by 6-hydroxydopamine, MPTP or MA is associated with increased levels of the tumor suppressor gene p53 (Duan et al., 2002; Biswas et al., 2005; Nair, 2006). Murine Double Minute 2 (MDM2) is an oncogene that mainly functions to modulate p53 activity. MDM2 ubiquitinates p53 and itself, leading to the degradation of both proteins (Haupt et al., 1997; Kubbutat et al., 1997).

Acetylsalicylic acid maltol ester [3-(2-methyl-4-pyronyl)-2-acetyloxybenzoate; AME] was synthesized by esterification of acetyl salicylic acid and maltol (Han et al., 1994). AME showed an antithrombotic efficacy with negligible gastrointestinal damage (Kim et al., 1997a) and an antioxidative efficacy in vitro (Han et al., 1994). Our previous studies indicated that the AME enhances antioxidant protection by elevating the activity of glutathione peroxidase (GPx) (Kim et al., 1996), and that AME attenuates neuroexcitotoxicity via antioxidant mechanism (Kim et al., 1997b).

Ebselen [2-phenyl-1,2-benzisoselenazol-3(2H)-one; EBS] is a synthetic seleno-organic compound, showing its antioxidative effect as a glutathione peroxidase mimic (Sies, 1993, Sies and Arteel, 2000). It has been used in the treatment of stroke due to its antioxidant and anti-inflammatory properties (Schewe, 1995; Yamaguchi et al., 1998; Lapchak and Zivin, 2003).

In the present study, we examined involvement of PKC in the MA-induced dopaminergic neurotoxicity. We observed that PKCδ out of PKC isozymes is important for contributing MA-toxicity. GPx-1 deficient mice was more prominent in inducing PKCδ. Our novel GPx mimic AME, well-known GPx mimic EBS or PKCδ inhibitor rottlerin enhanced GPx-1 expression as well as neurotrophic factors. Simultaneously they inhibit PKCδ and neuroinflammatory changes, oxidative stress. Thus, our novel GPx inducer AME, EBS and rottlerin may be potential candidates for blocking dopaminergic neurotoxicity.

The neurotoxin 1-methyl-4-phenyl-1,2,3,6-tetrahydropyridine (MPTP) is well known to cause the degeneration of nigrostriatal dopaminergic neurons with the loss of striatal dopamine (DA) (Heikkila et al., 1984) and decline of tyrosine hydroxylase (TH) levels in the substantia nigra pars compacta (SNpc) (Jakowec et al., 2004) in animals. MPTP is converted by monoamine oxidase-B (MAO-B) to 1-methyl-4-phenylpyridinium ion (MPP⁺) (Tipton and Singer., 1993), which is a neurotoxic metabolite and could block cellular respiration, promote reactive oxygen species (ROS) formation, and cause neuronal death (Olanow et al., 2006).

In the present study, it was examined whether an antioxidant, AME affects MPTP-induced dopaminergic toxicity. We observed that AME exerts a strong protective effect against MPTP-induced dopaminergic toxicity.

### SUMMARY OF THE INVENTION

Parkinson's disease (PD) is characterized by relatively selective nigrostriatal dopaminergic degeneration. 1-Methyl-4-phenyl-1,2,3,6-tetrahydropyridine (MPTP) is well known to damage the nigrostiatal dopaminergic neuron as seen in Parkinson's disease.

In one aspect, the invention is directed to a composition comprising a neuroprotective effective amount of acetylsalicylic acid maltol ester [3-(2-methyl-4-pyronyl)-2-acetyloxybenzoate] (AME) or an analog thereof or a physiologically acceptable salt thereof together with a pharmaceutical carrier or excipient. The composition may be in sustained release dosage form. The composition is directed to a Parkinson's disease symptom treatment effective amount.

In another aspect, the invention is directed to a unit dosage formulation for treatment of Parkinson's disease, comprising the composition described above or a pharmaceutically acceptable salt thereof in a form that is designed for oral ingestion by humans, wherein the 3-(2-methyl-4-pyronyl)-2-acetyloxybenzoate (AME) or an analog or salt thereof is present at a dosage which renders the 3-(2-methyl-4-pyronyl)-2-acetyloxybenzoate (AME) or an analog thereof therapeutically effective in substantially reducing symptoms of Parkinson's disease, without causing unacceptable side effects. The unit dosage formulation may include a digestible capsule. In one aspect, the dosage of the 3-(2-methyl-4-pyronyl)-2-acetyloxybenzoate (AME) or an analog thereof may be about 250 milligrams/day or less.

In another aspect, the invention is directed to a method of treating symptoms of Parkinson's disease comprising administering to a patient or animal in need of such treatment an effective anti-Parkinsonism amount of the composition described above. The composition may be in sustained release dosage form. The composition may also comprise a neuroprotective agent. The composition may include a digestible capsule, and may be administered at about 250 milligrams/day or less.

In still another aspect, the invention is directed to a method of preventing decrease of dopamine production in substantia nigra of a subject comprising administering to the subject a protective effective amount of the composition described above.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows experimental paradigm for evaluating methamphetamine-induced dopaminergic toxicity in mice. Methamphetamine (MA 8 mg/kg, i.p.) was administered four times as 2 hours′ time interval. RT=measurement of rectal temperature. Ambient temperature: 21±1° C.

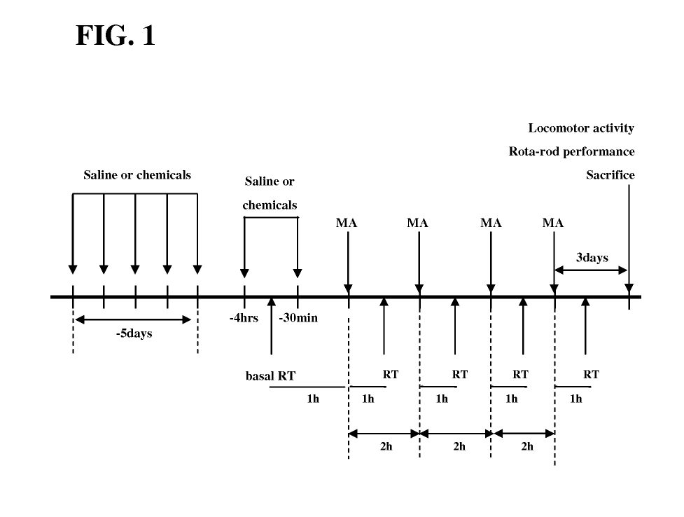

FIGS. 2A-2C show effects of PKC inhibitors on the MA-induced hyperthermia in GPx-1 (+/+)- and GPx-1 (−/−)-mice. MA=methamphetamine. CHE=chelerythrine chloride, pan-PKC inhibitor (A). Rot=rottlerin, PKC inhibitor (B). Go=Go6976, a co-inhibitor of PKCα and β. His=hispidin, PKCβ inhibitor. Zeta Inhi.=PKCξ pseudosubstrate inhibitor (C). Each value is mean±S.E.M. of 12 mice. Ambient temperature=21±1° C. ^{a}p<0.01 vs. respective Sal+Sal, ^{b}p<0.01 vs. respective Sal+MA, ^{c}p<0.05 vs. respective GPx-1 (+/+) mice (Statistics were performed by using ANOVA for repeated measures followed by Bonferroni's test). #=rectal temperature (° C.) of Sal+Sal-treated GPx-1 (+/+) mice is as follows; 36.6±0.40, 36.07±0.20, 36.72±0.25, 36.6±0.38 and 36.54±0.16 and that of Sal+Sal-treated GPx-1 (−/−) mice is as follows; 36.64±0.20, 36.68±0.20, 36.62±0.23, 36.57±0.18 and 36.41±0.17.

FIGS. 3A-3B show effects of PKC inhibitors on the locomotor activity (A) and rota-rod performance (B) induced by MA (8 mg/kg, i.p.×4) in GPx-1 (+/+)- and GPx-1 (−/−)-mice. CHE=chelerythrine chloride, pan-PKC inhibitor, Go6976=a co-inhibitor of PKCα and β, Hispidin=PKCβ inhibitor, PKCξ inhibitor=PKCξ pseudosubstrate inhibitor, Rot=rottlerin, PKCξ inhibitor. Each value is mean±S.E.M. of 12 mice. ap<0.05 vs. respective Sal+Sal, bp<0.05, bbp<0.01 vs. respective Sal+MA, cp<0.05 vs. respective GPx-1 (+/+) mice (Statistics were performed by using ANOVA followed by Fischer's PLSD test).

FIGS. 4A-4B show effects of chelerythrine or rottlerin on the PKCδ (A) and cleaved PKCδ (B) expressions induced by MA (8 mg/kg, i.p.×4) in GPx-1 (+/+)- and GPx-1 (−/−)-mice. CHE=chelerythrine chloride, pan-PKC inhibitor, Rot=rottlerin, PKCδ inhibitor. Each value is the mean±S.E.M of 6 mice. ap<0.01 vs. respective Sal+Sal, bp<0.05, bbp<0.01 vs. respective Sal+MA, cp<0.05 vs. respective GPx-1 (+/+) mice (Statistics were performed by using ANOVA followed by Fischer's PLSD test).

FIG. 5 shows information of acetylsalicylic acid maltol ester, ebselen and rottlerin. AME shows GPx mimic activities (Kim et al., 1996, 1997a), and prevented neuronal degeneration mainly via anti-peroxidative action (Kim et al 1007b). Dose and route: 25 or 50 mg/kg, p.o. EBS is an anti-inflammatory antioxidant as GPx mimetics (Sies, 1993; Sies and Arteel, 2000), and has been used in the treatment of stroke due to its antioxidant and anti-inflammatory properties (Schewe, 1995; Yamaguchi et al., 1998; Lapchak and Zivin, 2003). Dose and route: 10 or 20 mg/kg, p.o. Rottlerin, a natural compound from the medicinal tree Mallotus philippinensis, is used often as a specific inihibitor of PKCδ (Davies et al., 2000; Basu et al., 2001; Miller et al., 2007; Zhang et al., 2007). Dose and route: 10 or 20 mg/kg, p.o.

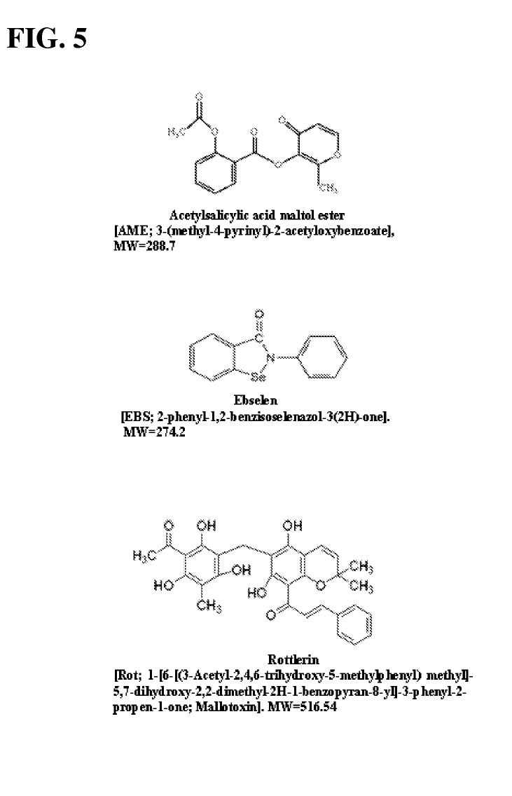

FIGS. 6A-6B show effects of orally-administered acetylsalicylic maltol ester (AME, 25 or 50 mg/kg), ebselen (EBS, 10 or 20 mg/kg) (A) or rottlerin (Rot, 10 or 20 mg/kg) (B) on the MA-induced hyperthermia in GPx-1 (+/+)- and GPx-1 (−/−)-mice. Each value is mean±S.E.M. of 12 mice. Ambient temperature=21±1° C. Sal=saline. MA=methamphetamine (8 mg/kg, i.p.×4). ap<0.01 vs. respective Sal+Sal, bp<0.01 vs. respective Sal+MA, cp<0.05 vs. respective GPx-1 (+/+) mice (Statistics were performed by using ANOVA for repeated measures followed by Bonferroni's test). #=rectal temperature (OC) of Sal+Sal-treated GPx-1 (+/+) mice is as follows; 37.4±0.25, 37.43±0.21, 37.3±0.15, 36.83±0.12 and 37.06±0.14; and that of Sal+Sal-treated GPx-1 (−/−) mice is as follows; 36.63±0.34, 36.7±0.05, 36.03±0.14, 36.56±0.14 and 36.23±0.16.

FIGS. 7A-7B show effects of orally-administered acetylsalicylic maltol ester (AME, 25 or 50 mg/kg), ebselen (EBS, 10 or 20 mg/kg) or rottlerin (Rot, 10 or 20 mg/kg) on the locomotor activity (A) and rota-rod performance (B) induced by MA (8 mg/kg, i.p.×4) in GPx-1 (+/+)- and GPx-1 (−/−)-mice. Each value is mean±S.E.M. of 12 mice. ap<0.05, aap<0.01 vs. respective Sal+Sal, bp<0.05 vs. respective Sal+MA, cp<0.05 vs. respective GPx-1 (+/+) mice (Statistics were performed by using ANOVA followed by Fischer's PLSD test).

FIGS. 8A-8C show effects of orally-administered acetylsalicylic maltol ester (AME, 25 or 50 mg/kg), ebselen (EBS, 10 or 20 mg/kg) or rottlerin (Rot, 10 or 20 mg/kg) on the PKCδ-(A), cleaved PKC δ-expressions (B) and the increases in PKCδ-like immunoreactivity (C) induced by MA (8 mg/kg, i.p.×4) in GPx-1 (+/+)- and GPx-1 (−/−)-mice. Sal=saline. Each value is the mean±S.E.M. of 6 animals for western blotting analysis. ap<0.01 vs. respective Sal+Sal, bp<0.05, bbp<0.01 vs. respective Sal+MA, cp<0.05 vs. respective GPx-1 (+/+) mice (Statistics were performed by using ANOVA followed by Fischer's PLSD test).

FIGS. 9A-9C show effects of orally-administered acetylsalicylic maltol ester (AME, 50 mg/kg), ebselen (EBS, 20 mg/kg) or rottlerin (Rot, 20 mg/kg) on the striatal glutathione peroxidase (GPx)-1-IR (A, B), and GPx-1 activity (C) induced by MA (8 mg/kg, i.p.×4) in GPx-1 (+/+) mice. Each value is mean±S.E.M. of 6 mice. ap<0.01 vs. respective Sal+Sal, bp<0.05, bbp<0.01 vs. respective Sal+MA (Statistics were performed by using ANOVA followed by Fischer's PLSD test).

FIGS. 10A-10E show effects of orally-administered acetylsalicylic maltol ester (AME, 50 mg/kg), ebselen (EBS, 20 mg/kg) or rottlerin (Rot, 20 mg/kg) on the values of dopamine (DA; A), 3,4 dihydroxyphenylacetic acid (DOPAC; B), homovanillic acid (HVA; C), DA turnover rate (D), and the reduction of tyrosine hydroxylase activity (TH; E) induced by MA (8 mg/kg, i.p.×4) in GPx-1 (+/+)- and GPx-1 (−/−)-mice. Each value is the mean±S.E.M. of 8 animals. ap<0.05, aap<0.01 vs. respective Sal+Sal, bp<0.05, bbP<0.01 vs. respective Sal+MA, cp<0.05 vs. respective GPx-1 (+/+) mice (Statistics were performed by using ANOVA followed by Fischer's PLSD test).

FIGS. 11A-11B show effects of orally-administered acetylsalicylic maltol ester (AME, 50 mg/kg), ebselen (EBS, 20 mg/kg) or rottlerin (Rot, 20 mg/kg) on the striatal tyrosine hydroxylase-like immunoreactivity (TH-IR) (A) and the substantia nigral pars compacta tyrosine hydroxylase-like immunoreactivity (TH-IR) (B) induced by MA (8 mg/kg, i.p.×4) in GPx-1 (+/+)- and GPx-1 (−/−)-mice. Each value is the mean±S.E.M. of 6 mice. ap<0.01 vs. respective Sal+Sal, bp<0.01 vs. respective Sal+MA, cp<0.05 vs. respective GPx-1 (+/+) mice (Statistics were performed by using ANOVA followed by Fischer's PLSD test).

FIGS. 12A-12C show effects of orally-administered acetylsalicylic maltol ester (AME, 50 mg/kg), ebselen (EBS, 20 mg/kg) or rottlerin (Rot, 20 mg/kg) on the striatal tyrosine hydroxylase—like immunoreactivity (TH-IR) induced by MA (8 mg/kg, i.p.×4) in GPx-1 (+/+)- and GPx-1 (−/−)-mice [Western blot for PAN-TH (A), TH phospho-ser 31 (B), and TH phospho-ser 40 (C)]. Each value is the mean±S.E.M. of 6 mice. ap<0.05, aap<0.01 vs. respective Sal+Sal, bp<0.05 vs. respective Sal+MA, cp<0.05 vs. respective GPx-1 (+/+) mice (Statistics were performed by using ANOVA followed by Fischer's PLSD test).

FIGS. 13A-13B show effects of orally-administered acetylsalicylic maltol ester (AME, 50 mg/kg), ebselen (EBS, 20 mg/kg) or rottlerin (Rot, 20 mg/kg) on striatal expressions of p53 [A], MDM2 and phospho MDM2-ser 166 [B] induced by MA (8 mg/kg, i.p.×4) in the GPx-1 (+/+)- and GPx-1 (−/−)-mice. Each value is the mean±S.E.M. of 6 mice. ap<0.01 vs. respective Sal+Sal, bp<0.01 vs. respective Sal+MA, cp<0.05 vs. respective GPx-1 (+/+) mice (Statistics were performed by using ANOVA followed by Fischer's PLSD test).

FIGS. 14A-14F show effects of orally-administered acetylsalicylic maltol ester (AME, 50 mg/kg), ebselen (EBS, 20 mg/kg) or rottlerin (Rot, 20 mg/kg) on the COX-2-(A), F4/80-(B), Iba-1-(C), IL-6-(D), TNF-α-(E), and IFN-γ-expressions (F) induced by MA (8 mg/kg, i.p.×4) in GPx-1 (+/+)- and GPx-1 (−/−)-mice. Sal=saline. Each value is the mean±S.E.M. of 6 mice. ap<0.01 vs. respective Sal+Sal, bp<0.05 vs. respective Sal+MA, cp<0.05 vs. respective GPx-1 (+/+) mice (Statistics were performed by using ANOVA followed by Fischer's PLSD test).

FIGS. 15A-15B show effects of orally-administered acetylsalicylic maltol ester (AME, 50 mg/kg), ebselen (EBS, 20 mg/kg) or rottlerin (Rot, 20 mg/kg) on the striatal expressions of neurotrophic factors induced by MA (8 mg/kg, i.p.×4) in the GPx-1 (+/+)- and GPx-1 (−/−)-mice. BDNF=brain-derived neurotrophic factor (A). GDNF=glial cell line-derived neurotrophic factor (B). Sal=saline. Each value is the mean±S.E.M. of 6 mice. ap<0.05, aap<0.01 vs. respective Sal+Sal, bp<0.05 vs. respective Sal+MA, cp<0.05 vs. respective GPx-1 (+/+) mice (Statistics were performed by using ANOVA followed by Fischer's PLSD test).

FIGS. 16A-16C show effects of orally-administered acetylsalicylic maltol ester (AME 50 mg/kg), ebselen (EBS, 20 mg/kg) or rottlerin (Rot, 20 mg/kg) on the MA-induced formation of reactive oxygen species (ROS) (A), lipid peroxidation (B), protein oxidation and (C), 3 days after the final MA administration of GPx-1 (+/+)- and GPx-1 (−/−)-mice. MDA=malondialdehyde. 4-HNE=4-hydroxynonenal. Each value is mean±S.E.M. of 8 mice. ap<0.01 vs. respective Sal+Sal, bp<0.05 vs. respective Sal+MA, cp<0.05 vs. respective GPx-1 (+/+) mice (Statistics were performed by using ANOVA followed by Fischer's PLSD test).

FIG. 17 shows flow chart describing our current hypothesis on the roles of GPx-1 gene and PKCδ gene in the MA-induced dopaminergic neurotoxicity.

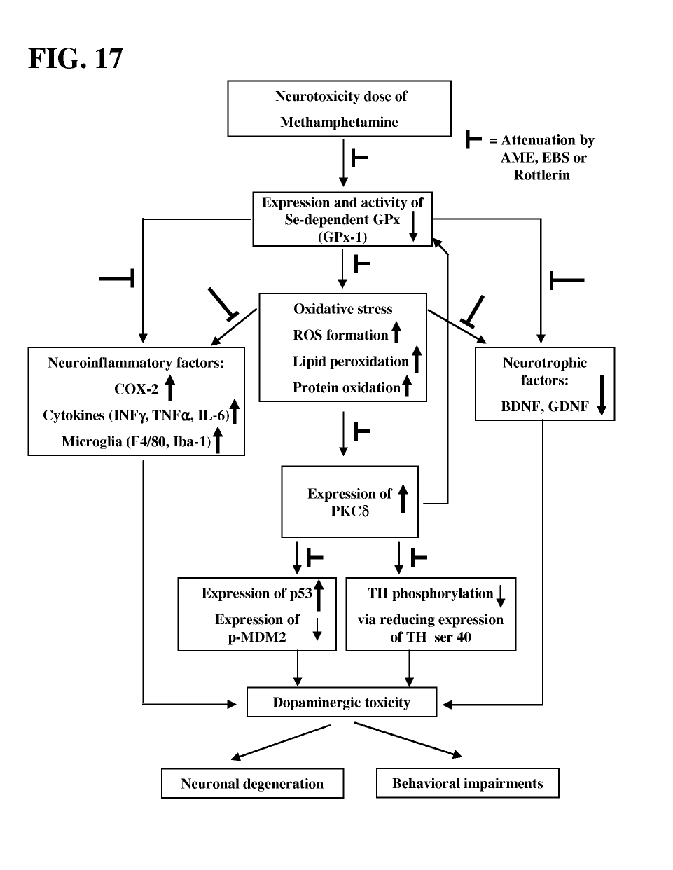

FIG. 18 shows AME (25 mg/kg, p.o) administration was started for 4 days (twice daily) before the first injection of MPTP and continued for 7 consecutive days (once a day). MPTP was injected (25 mg/kg, i.p) once a day 1 h after AME treatment for 7 days.

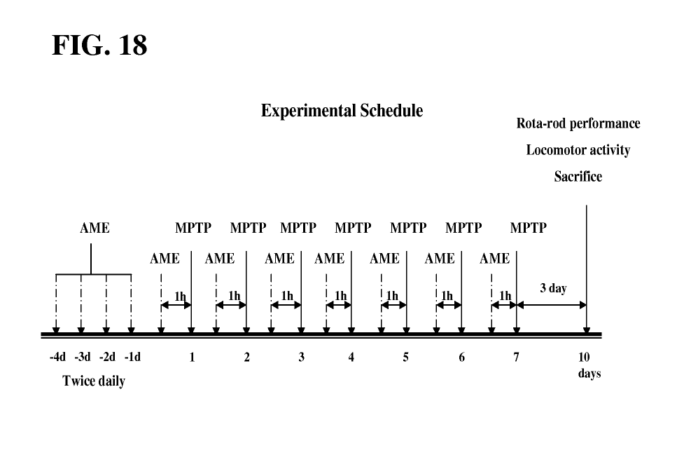

FIGS. 19A-19B show effects of AME (25 mg/kg, p.o.) on the changes in locomotor activity (A) and rota-rod performance (B) 3 days after final treatment with MPTP in mice. Each value is the mean±S.E.M. of 10 animals. *P<0.05 vs. Saline+Saline, ^{#}P<0.05 vs. Saline+MPTP (ANOVA with Fisher's PLSD test).

FIGS. 20A-20D show shows effects of AME (25 mg/kg, p.o.) on MPTP-induced changes in dopamine (DA; A), 3,4-dihydroxyphenylacetic acid (DOPAC; B), homovanillic acid (HVA; C) and DA turnover rate (D) in the striatum of the mice. Each value is the mean±S.E.M. of 7 animals. *P<0.01 vs. Saline+Saline, ^{#}P<0.05 vs. Saline+MPTP, ^{##}P<0.01 vs. Saline+MPTP (ANOVA with Fisher's PLSD test).

FIGS. 21A-21B show effects of AME on the MPTP-induced striatal (A) and nigral (B) decreases in tyrosine hydroxylase-like immunoreactivity (TH-IR). Each value is the mean±S.E.M. of 4 animals. *P<0.01 vs. Saline+Saline, ^{#}P<0.01 vs. Saline+MPTP (ANOVA with Fisher's PLSD test).

### DETAILED DESCRIPTION OF THE PREFERRED EMBODIMENTS

In the present application, “a” and “an” are used to refer to both single and a plurality of objects.

As used herein, “effective amount” is an amount sufficient to effect beneficial or desired clinical or biochemical results. An effective amount can be administered one or more times. For purposes of this invention, an effective amount of a AME analog compound is an amount that is sufficient to palliate, ameliorate, stabilize, reverse, slow or delay the progression of a disease state or condition. In a preferred embodiment of the invention, the “effective amount” is defined as an amount of compound capable of preventing decrease in formation of dopamine in substantia nigra, and is an amount that substantially reduces the symptoms of Parkinson's disease. Other forms of effective amount may be for the treatment or prevention of the learning or memory impairment related to Alzheimer's disease. In yet another embodiment, the “effective amount” is defined as the neuroprotective effective amount of the AME analog compound.

As used herein, administration “in combination with” one or more further therapeutic agents includes simultaneous (concurrent) and consecutive administration in any order.

As used herein, “mammal” or “subject” for purposes of treatment refers to any animal classified as a mammal, including humans, domestic and farm animals, and zoo, sports, or pet animals, such as dogs, cats, cattle, horses, sheep, pigs, and so on. Preferably, the mammal is human.

As used herein, “neuroprotective” agent refers to drugs or chemical agents intended to prevent damage to the brain or spinal cord from ischemia, stroke, convulsions, or trauma. Some must be administered before the event, but others may be effective for some time after. They act by a variety of mechanisms, but often directly or indirectly minimize the damage produced by endogenous excitatory amino acids. Neuroprotection also includes protection against neurodegeneration and neurotoxins. Further, by “neuroprotective” it is meant to include intervention that slows or halts the progression of neuronal degeneration. Neuroprotection may also be used for prevention or progression of a disease if it can be identified at a presymptomatic stage.

As used herein, “Parkinson's disease” refers to a chronic progressive nervous disease chiefly of later life that is linked to decreased dopamine production in the substantia nigra. Symptoms include stooped posture, resting tremor, weakness of resting muscles, a shuffling gait, speech impediments, movement difficulties and an eventual slowing of mental processes and dementia.

As used herein, “3-(2-methyl-4-pyronyl)-2-acetyloxybenzoate (AME) analog” may be any variant of AME that has an anti-Parkinsonian effect. The AME analog attenuates MPTP-induced toxicity.

Therapeutic Formulations

Administration of the AME compound and its analogs and their mixtures and/or pharmaceutically acceptable salts can be orally or transdermally or by intravenous, intramuscular, subcutaneous, intrathecal, epidural or intracerebro-ventricular injection. Effective dosage levels can vary widely, e.g., from about 0.25 to about 250 mg/day, but actual amounts will, of course, depend on the state and circumstances of the patient being treated. As those skilled in the art recognize, many factors that modify the action of the active substance herein will be taken into account by the treating physician such as the age, body weight, sex, diet and condition of the patient, the time of administration, the rate and route of administration, and so forth. Optimal dosages for a given set of conditions can be ascertained by those skilled in the art using conventional dosage determination tests in view of the experimental data provided herein.

Therapeutic compositions containing the AME compound and its analogs, their mixtures and/or pharmaceutically acceptable salts will ordinarily be formulated with one or more pharmaceutically acceptable ingredients in accordance with known and established practice. Thus, the AME compound and its analogs, their mixtures and/or pharmaceutically acceptable salts can be formulated as a liquid, powder, elixir, injectable solution, etc. Formulations for oral use can be provided as hard gelatin capsules wherein the AME compound and its analogs, their mixtures and/or pharmaceutically acceptable salts are mixed with an inert solid diluent such as calcium carbonate, calcium phosphate or kaolin, or as soft gelatin capsules wherein the AME compound and its analogs, their mixtures and/or pharmaceutically acceptable salts are mixed with an oleaginous medium, e.g., liquid paraffin or olive oil.

Aqueous suspensions can contain the AME compound and its analogs, their mixtures and/or pharmaceutically acceptable salts in admixture with pharmaceutically acceptable excipients such as suspending agents, e.g., sodium carboxymethyl cellulose, methylcellulose, hydroxypropylmethylcellulose, sodium alginate, polyvinylpyrrolidone, gum tragacanth and gum acacia; dispersing or wetting agents such as naturally occurring phosphatide, e.g., lecithin, or condensation products of an alkaline oxide with fatty acids, e.g., polyoxyethylene stearate, or condensation products of ethylene oxide with long chain aliphatic alcohols, e.g, heptadecaethylene-oxycetanol, or condensation products of ethylene oxide with partial esters derived from fatty acids and a hexitol, e.g., polyoxyethylene sorbitol monoleate or condensation products of ethylene oxide with partial esters derived from fatty acids and hexitol anhydrides, e.g., polyoxyethylene sorbitan monoleate. Such aqueous suspensions can also contain one or more preservatives, e.g., ethyl-or-n-propyl-p-hydroxy benzoate, one or more coloring agents, one or more flavoring agents and one or more sweetening agents, such as sucrose, saccharin or sodium or calcium cyclamate.

Dispersible powders and granules suitable for preparation of an aqueous suspension by the addition of water provide the AME compound and its analogs, their mixtures and/or pharmaceutically acceptable salts in admixture with a dispersing or wetting agent, suspending agent and one or more preservatives. Suitable dispersing or wetting agents and suspending agents are exemplified by those already mentioned above. Additional excipients, e.g., sweetening, flavoring and coloring agents, can also be present. Syrups and elixirs can be formulated with sweetening agents, for example glycerol, sorbitol or sucrose. Such formulations can also contain a demulcent, a preservative and flavoring and coloring agents.

The AME compound and its analogs, their mixtures and/or pharmaceutically acceptable salts are advantageously provided in sustained release dosage form of which many kinds are known, e.g., as described in U.S. Pat. Nos. 4,788,055; 4,816,264; 4,828,836; 4,834,965; 4,834,985; 4,996,047; 5,071,646; and, 5,133,974, the contents of which are incorporated by reference herein.

It is also within the scope of this invention to administer the AME compound and its analogs, their mixtures and/or pharmaceutically acceptable salts prior to, concurrently with, or after administration of any other known pharmacologically active agent useful for treating or treating the symptoms of Parkinson's disease. Such pharmacologically active agents may include without limitation other neuroprotective agents.

Neuroprotective agents attempt to save ischemic neurons in the brain from irreversible injury. Other neuroprotective agents prevent potentially detrimental events associated with return of blood flow. Although return of blood flow to the brain is generally associated with improved outcome, reperfusion may contribute to additional brain injury. Returning blood contains leukocytes that may occlude small vessels and release toxic products. Ischemia leads to excessive activation of excitatory amino acid receptors, accumulation of intracellular calcium, and release of other toxic products that cause cellular injury. By preventing excitatory neurotransmitter release, neuroprotective agents may reduce deleterious effects of ischemia on cells.

Instructions

The present invention is also directed to instructions regarding the use the inventive AME compound and its analogs, for treating a variety of neurological conditions, including Parkinson's disease or the symptoms of Parkinson's disease, learning and memory impairment in Alzheimer's disease. Such instructions may be in a permanent or temporary format. The instructions may be in written form, such as but not limited to a textbook, protocol book, catalog, internet web site and so on. Such instructions may be in relation to but not limited to the sale and use of the AME compound and its analogs. The instructions may be presented via a computer screen on a cathode ray tube, LCD, LED, and so on, so long as the instructions are visible through the eye. The instructions may also be in the form of audio/visual media, or as part of a kit for treating the various symptoms as indicated above.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Material and Methods

### Example 1.1

### Animal

All mice were treated in strict accordance with the NIH Guide for the Humane Care and Use of Laboratory Animals. They were maintained on a 12 h light: 12 dark cycle and fed ad libitum. Also, they were adapted to these conditions for 2 weeks before the experiment. GPx-1 (−/−) mice used in this study have been described by Ye-Shih et al. (1997), previously. PCR analyses using DNA templates extracted from the mouse tails were performed for characterization.

### Example 1.2

### Drug Treatments

The mice received four times of MA (8 mg/kg, ip) or saline as a 2 hr-time interval. Chemicals (AME, EBS, and PKC inhibitors) were administrated for 5 consecutive days (twice daily), and were given 2 times at 4 hr and 30 min before the first MA injection as shown in the experimental schedule. Animals were sacrificed at 4 hrs and 3 days after the final MA administration.

### Example 1.3

### Temperature Measurement

Rectal temperature was measured in the MA- or saline-treated mice. Measurement was performed at constant daytime intervals starting at 9:00 A.M. to avoid the influence on circadian variations. Rectal temperature was measured by inserting a thermometer probe lubricated with oil at least 3 cm into the rectum of the mice. To prevent sudden movements occurring especially in MA-treated mice, animals were gently handled with a wool glove while their tail was moved to allow the probe insertion. This was done to prevent the effects of restrain stress on rectal temperature. When the attempt to insert probe was not successful (i.e., sudden movements of the animal or the need to restrain the mouse), the animal was excluded from the groups.

### Example 1.4

### Locomotor Activity

Locomotor activity was measured for 30 min 3 days after the last MA administration using an automated video-tracking system (Noldus Information Technology, Wagenin, The Netherlands). Four test boxes (40×40×30 cm high) were operated simultaneously by an IBM computer. Mice were studied individually during locomotion in each test box, where they were adapted for 5 min before starting the experiment. A printout for each session showed the pattern of the ambulatory movements of the test box. The distance traveled in cm by the animals in horizontal locomotor activity was analyzed. Data were collected and analyzed between 09:00 and 17:00 h (Kim et al., 2001).

### Example 1.5

### Rota-Rod Test

The apparatus (Ugo Basile model 7650, Comerio, VA, Italy) consisted of a base platform and a rotating rod with a nonslippery surface. The rod was placed at a height of 15 cm from the base. The rod, 30 cm in length, was divided into 5 equal sections by 6 opaque disks (so that the subjects cannot be distracted by one another) To assess motor performance, the mice first trained on the apparatus 2 minutes at a constant rate of 4 r.p.m. per 30 s prior to the test. The test was performed 30 minutes after training and an accelerating paradigm was applied, starting from a rate of 4 r.p.m. to a maximum speed of 40 r.p.m., then the rotation speed was kept constant at 40 r.p.m. for a maximum of 300 s. The duration for which the animal could maintain balance on the rotating drum was measured as the rotarod latency, with a maximal cut-off time of 300 s.

### Example 1.6

### Immunocytochemistry

Animals were sacrificed at 4 hours and 3 days after the last MA injection. They were anesthetized with 60% urethane and perfused transcardially with 50 ml of 50 mM phosphate buffered saline (PBS), followed by 50 ml of a mixture of 4% paraformaldehyde in PBS. The rate of perfusion was 50 ml/min. The brains were removed, post-fixed at 4° C. for 24 h in the same fixative and then cryoprotected in 30% sucrose in PBS. The brains were cut on a horizontal sliding microstome into 40 μm transverse free-floading sections (Kim et al., 1999).

The immunocytochemistry was performed as described previously (Kim et al., 2000a, b). Briefly, prior to incubation with the primary antibodies, sections were preincubated with 0.3% hydrogen peroxide in PBS for 30 min (to block endogenous peroxidase activity), then in PBS containing 0.4% Triton X-100 for 20 min and 1% normal serum for 20 min. The sections were then incubated for 48 h at 4° C. in primary antibody against tyrosine hydroxylase (1:500, Chemicon, Termecula, Calif., USA), GPx1 (1:500, AbFrontier, Seoul, Korea), PKC (1:200, Santa Cruz biotechnology INC, CA, USA) or F4/80 (1:50, Serotec, Raleigh, N.C., USA). The sections were further incubated with secondary biotinylated antisera (1:1000 dilution; Vector, Brulingame, Calif.) for 1 hr, and immersed in avidin-biotin-peroxidase complex (ABC Elite kit, Vector) for 1 hr. Sections were always washed three times with PBS (pH 7.4) between each incubation step. 3,3′-diaminobenzidine (DAB) was used as a chromogen.

### Example 1.7

### Western Blot Analysis

The western blot assays was performed as described previously (Kim et al., 2003). Tissues were homogenized in lysis buffer, containing 200 mM Tris HCl (pH 6.8), 1% SDS, 5 mM EGTA (ethylene glycol tetraacetic acid), 5 mM EDTA (ethylenediaminetetraacetic acid), 10% glycerol, 1× phosphatase inhibitor cocktail I, 1× protease inhibitor cocktail. Lysate was centrifuged at 12,000×g for 30 min and supernatant fraction was used for Western blot analysis. Proteins (20-50 ug/lane) were separated by 6%, 8%, 10% or 15% sodium dodecyl sulfate-polyacrylamide gel electrophoresis and transferred onto the PVDF membranes. Following transfer, the membranes were preincubated with 5% non-fat milk and incubated overnight at 4° C. with primary antibody against β-actin (1:50000, Sigma, St. Louis, Mo., USA), TH (1:5000, Chemicon), TH phosphor-Ser 19 (1:1000, Chemicon,), TH phosphor-Ser 31 (1:500, Chemicon), TH phosphor-Ser 40 (1:500, Chemicon), F4/80 (1:500, Serotec), GPx1 (1:2500, AbFrontier), PKC (1:5000, Santa Cruz biotechnology, Santa Cruz, Calif., USA), p53 (1:5000, Cell signaling, Beverly, Mass., USA), MDM2 (1:1000, BD Pharmingen, San Jose, Calif., USA), MDM2 phospho-Ser 166 (1:1000, cell signaling), BDNF (1:500, Chemicon), GDNF (1:250, Santa Cruz), COX-2 (1:2000, Cayman, Ann Arbor, MC, USA), IL-6 (1:2000, Abcam, Cambridge, Mass., USA), TNF-a (1:1000, R&D Systems, Mckinley place NE, NM, USA), IFN-γ (1:500, Chemicon) or Iba-1 (1:500, Wako, Osaka, Japan). And then, membranes were incubated with HRP-conjugated secondary anti-rabbit IgG (1:1000, GE healthcare, Piscataway, N.J., USA), anti-mouse IgG (1:1000, Sigma) or anti-goat IgG (1:1000, Sigma) for 2 h. Subsequent visualization was performed using enhanced chemiluminescence system (ECL Plus®, GE healthcare).

### Example 1.8

### Measurement of Dopamine (DA), 3,4-dihydroxyphenylacetic Acid (DOPAC) and Homovanillic (HVA)

At 3 day after last MA injection, mice were killed by cervical dislocation and the brains were removed. Striatum was dissected and immediately frozen on dry ice, and stored at −70° C. until extraction. Striatum obtained from each animal was weighed, ultrasonicated in 10% perchloric acid containing 10 ng/mg of the internal standard dihydroxybenzylamine, and centrifuged at 20,000×g for 10 min. The levels of DA and its metabolites 3,4-dihydroxyphenylacetic acid (DOPAC) and homovanillic acid (HVA) in brain tissue extracts were determined by HPLC coupled with electrochemical detection as described (Kim et al., 1999). The 20 μl aliquot of the supernatant was injected into the HPLC equipped with a 3 μm C18 column. The mobile phase was comprised of 26 ml of acetonitrile, 21 ml of tetrahydrofuran and 960 ml of 0.15M monochloroacetic acid (pH 3.0) containing 50 mg/l of EDTA and 200 mg/l of sodium octyl sulfate. The amount of DA, DOPAC and HVA were determined by comparison of peak area of tissue sample with standard, and were expressed in nanograms per gram of wet tissue.

### Example 1.9

### Measurement of Tyrosine Hydroxylase (TH) Activity

TH activity was measured according to the method of Lucock et al. with some modification (Duan et al., 2005). Briefly, striatum was lysed in 400 μl TH working solution (1-tyrosine: 300 μmol/L; FeSO₄: 1 mmol/L; NaAC: 200 mmol/L; NSD-1050:500 won; DTT: 1 mmol/L; MES: 40 mmol/L, pH 5.2-5.6) with freezing-thawing repeatedly for three times. The lysate was reacted for 3 h at 25° C. The reaction was stopped by 0.4 mol/L perchloric acid, and then reactant was centrifuged at 14,000×g for 10 min. Supernatants were collected to assay the amounts of 1-dopa by HPLC-ECD. The activity of TH was expressed as that amount of 1-dopa per minute and per gram of wet tissue.

### Example 1.10

### Determination of ROS Formation

The extent of reactive oxygen species (ROS) formation in the striatum was assessed by measuring the convertion from 2′,7′-dichlorofluorescin diacetate (DCFH-DA) to dichlorofluoresin (DCF) as describe by Bourre et al., (2002) with slight modification. Brain homogenates were added to a tube containing 2 ml of PBS with 10 nmole of DCFH-DA, dissolved in methanol. Mixture were incubated at 37° C. for 3 hours and then measured the absorbance at 480 nm excitation and 525 nm emission. DCF is used as a standard.

### Example 1.11

### Determination of Protein Oxidation

### Example 1.11.1

### Determination of Protein Carbonyl (Oxyblot Assay)

The amount of oxidized proteins was measured using the Oxyblot kit (Chemicon International, CA) according to the instruction provided by manufacturer. Briefly, the protein carbonyl content was measured by first forming labeled protein hydrazone derivatives using 2,4-dinitrophenylhydrazide (DNP). The DNP-derivatized protein samples were transferred onto PVDF membrane by slot blot apparatus (GE Healthcare). Blots were then incubated with primary antibody specific to the DNP moiety, followed by incubation with a HRP-conjugated secondary antibody. Subsequent visualization was performed using enhanced chemiluminescence system (ECL Plus®, GE healthcare) (Gemma et al., 2004).

### Example 1.11.2

### Determination of Protein Carbonyl (Biochemical Assay)

The extent of protein carbonyl oxidation in the striatum was assessed by measuring the content of protein carbonyl groups, which was determined spectrophotometrically with the 2,4-dinitrophenyl-hydrazine (DNPH)-labeling procedure (Kim et al., 1997b, 2000b, 2002) as described by Oliver et al. (1987). The results were expressed as nmol of DNPH incorporated/mg protein based on the extinction coefficient for aliphatic hydrazones of 21 mM⁻¹ cm⁻¹. Protein was measured using the BCA protein assay reagent (Pierce, Rockford, Ill., USA).

### Example 1.12

### Determination of Lipid Peroxidation

### Example 1.12.1

### Determination of Malondialdehyde (MDA)

The amount of lipid peroxidation was assessed by measuring the accumulation of thiobarbituric acid-reactive substance in homogenates of striatal tissue and is expressed as malondialdehyde (MDA) content (Kim et al., 1999, 2000b, 2002). In brief, 0.1 ml of the homogenate (or standard solutions prepared daily from 1,1,3,3-tetra-methoxypropane) and 0.75 ml of the working solution (thiobarbituric acid 0.37% and perchloric acid 6.4%, 2:1 v/v) were mixed and heated in a water bath to 95° C. for 1 h. After cooling (10 min in ice water bath), the flocculent precipitate was removed by centrifugation at 3200×g for 10 min. The supernatant was neutralized and filtered prior to injection on an ODS 5 μm column. Mobile phase consisted of 50 mM PBS (pH 6.0): methanol (58:42, v/v). Isocratic separation was performed with 1.0 ml/min flow rate and detection at 532 nm using a UV/VIS HPLC-Detector (Model 486, Waters Associates, Milford, Mass., USA). MDA values were expressed as nmole/mg protein.

### Example 1.12.2

### Determination of 4-hydroxy-2-nonenal (4HNE)

4-hydroxy-2-nonenal (4HNE) is major product of the lipid peroxidation process (Benedetti et al., 1980). Determining of 4HNE was performed as slot blot analysis (Zhang et al., 2000). Briefly, following transfer, the PVDF membranes were preincubated with 5% non-fat milk and incubated overnight at 4° C. with anti-4HNE (1:2000, Calbiochem, San Diego, Calif., USA). After incubation with primary antibody, membranes were incubated with a HRP-conjugated secondary antibody. Subsequent visualization was performed using enhanced chemiluminescence system (ECL Plus®, GE healthcare).

### Example 1.13

### Measurement of Glutathione Peroxidase (GPx) Activity

GPx were immediately measured from dissected striatal tissues. The tissues were sonicated in ice-cold 50 mM potassium phosphate buffer (pH 7.4, containing 2 mM EDTA), and were centrifuged at 11,000×g for 15 min at 4° C. The resulting supernatants were collected and the protein concentrations were quantified with the Quant-iT assays, using the Qubit™ fluorometer (Invitrogen, Carlsbad, Calif., USA). Cellular GPx activity was measured by the method of Paglia and Valentine (1967) with minor modification (Shin et al., 2008). The incubation mixture contained 1 mM glutathione, 0.2 mM NADPH, and 1.4 IU glutathione reductase in 0.05 M potassium phosphate buffer, pH 7.0. The reaction was initiated by the simultaneous addition of supernatant (0.3-0.8 mg protein) and 0.25 mM H₂O₂. The change in absorbance at 340 nm was followed by for 4.5 min and 1 UI of GPx activity was defined as the amount required to oxidize 1 μM NADPH/min, based on the molar absorptivity of 6.22×10⁻⁶ for NADPH.

### Example 1.14

### Statistical Analysis

The data were analyzed using a one-way ANOVA followed by Fischer's PLSD test or a two-way ANOVA for repeated measures followed by Bonferroni's test. p values of less than 0.05 were deemed statistically significant.

### Example 2

### Results

### Example 2.1

### Chelerythrine (PAN-Inhibitor of PKC) or Rottlerin (Inhibitor of PKCδ), but not any Other Inhibitors of PKC Isoforms, Attenuated MA-Induced Hyperthermia in the GPx-1 (+/+)- and GPx-1 (−/−)-Mice

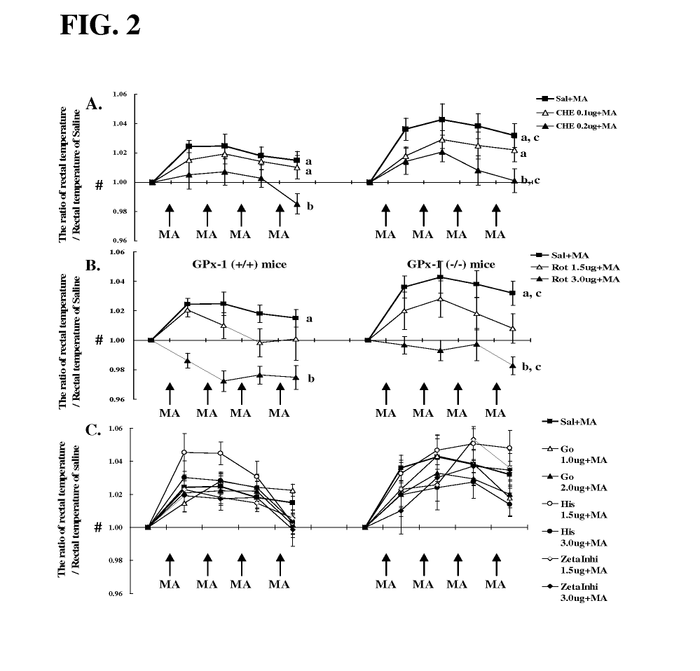

Treatment with MA produced hyperthermia in the GPx-1 (+/+)- and GPx-1 (−/−)-mice (p<0.01 vs. each strain of saline-treated mice). MA-induced hyperthermia was more pronounced in GPx-1 (−/−) mice (p<0.05) than GPx-1 (+/+) mice. Treatment with chelerythrine (CHE), a PAN-PKC inhibitor, or rottlerin, a PKC inhibitor, significantly blocked MA-induced hyperthermia in a dose-dependent manner (p<0.01 vs. each strain of MA-treated mice). Treatment with inhibitors of any other PKC isoforms, such as, Go 6976, PKCa and PKCβ inhibitor; hispidin, PKCβ inhibitor; and PKCξ pseudosubstrate inhibitor, did not show any significant effect on the MA-induced hyperthermia in each strain of mice (FIG. 2).

### Example 2.2

### Chelerythrine (PAN-Inhibitor of PKC) or Rottlerin (Inhibitor of PKCδ), but Not any Other Inhibitors of PKC Isoforms, Attenuated MA-Induced Behavioral Impairments in the GPx-1 (+/+)- and GPx-1 (−/−)-Mice

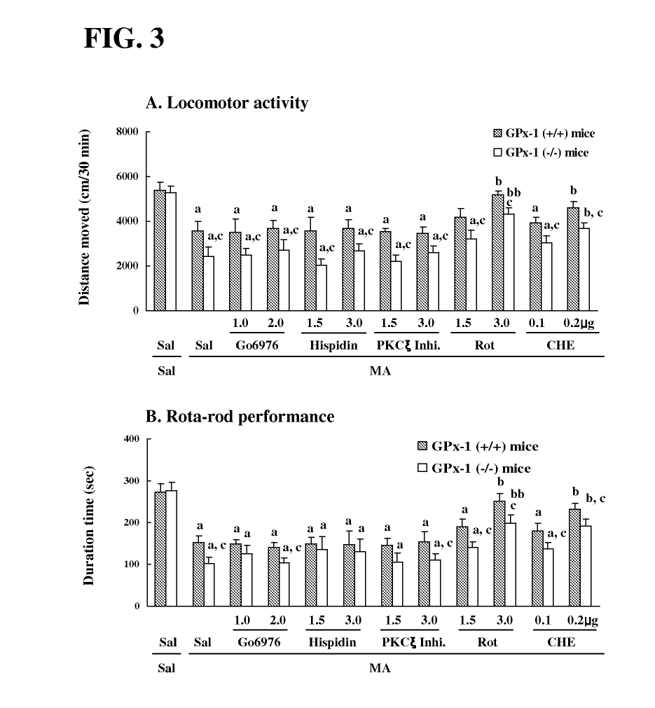

Treatment with MA produced impairments in the locomotor activity and rota-rod performance (p<0.05 vs. each strain of saline-treated mice) (FIG. 3). These impairments were more significant in GPx-1 (−/−) mice (p<0.01) than GPx-1 (+/+) mice. Treatment with chelerythrine, PAN-PKC inhibitor, or rottlerin, PKCδ inhibitor, attenuated the MA-behavioral impairments in a dose-dependent manner [chelerythrine 0.2 ug/head: p<0.05 vs. MA-treated mice; rottlerin 3.0 ug/head: p<0.05 vs. MA-treated GPx-1 (+/+), p<0.01 vs. MA-treated GPx-1 (−/−)]. Treatment with any other inhibitor of PKC isoforms (such as, Go 6976, PKCα and PKClβ inhibitor; hispidin, PKCβ inhibitor; and PKC zeta pseudosubstrate inhibitor) did not affect MA-induced behavioral impairments.

### Example 2.3

### Chelerythrine or Rottlerin Attenuated the MA-Induced Alterations in PKCδ Expressions in the GPx-1 (+/+)- and GPx-1 (−/−)-Mice

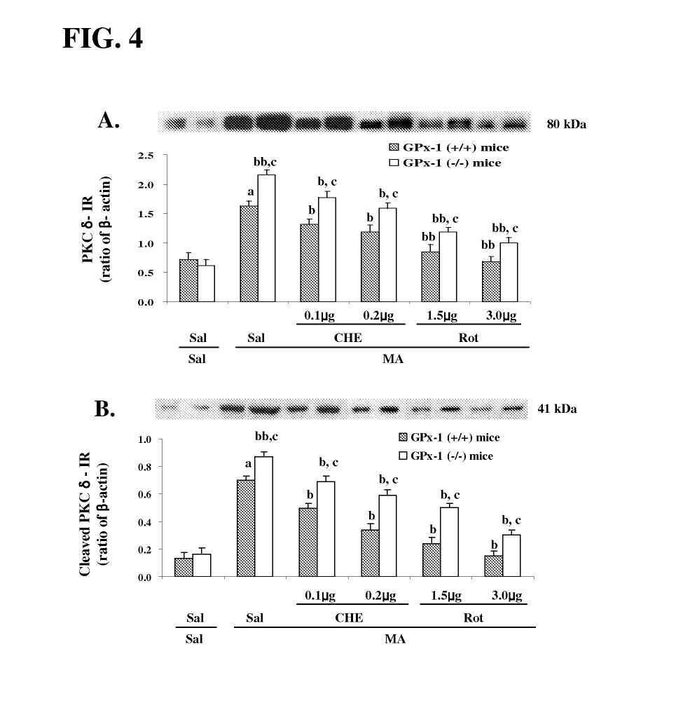

MA-induced increase in the striatal expression of PKCδ and cleaved PKCδ (an active form of PKCδ) was observed (FIG. 4). This finding was more pronounced in GPx-1 (−/−) mice (p<0.05) than GPx-1 (+/+) mice. Treatment with chelerythrine or rottlerin attenuated the MA-induced increases in PKCδ expressions in a dose-dependent manner in each strain of mice [chelerythrine: p<0.05 vs. MA-treated mice; rottlerin: PKCδ-IR, p<0.01 vs. MA-treated mice; cleaved PKCδ-IR, p<0.05 vs. MA-treated mice].

### Example 2.4

### Orally-Administered Acetylsalicylic Acid Maltol Ester (AME), Ebselen (EBS), or Rottlerin Attenuated the MA-Induced Hyperthermia in the GPx-1 (+/+)- and GPx-1 (−/−)-Mice

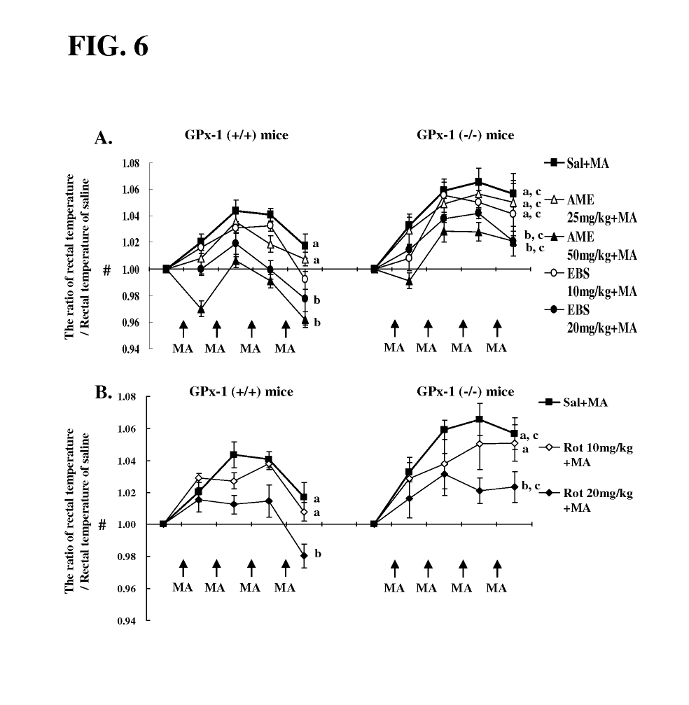

MA significantly induced hyperthermia in the GPx-1 (+/+)- and GPx-1 (−/−)-mice (p<0.01 vs. each strain of saline-treated mice). Treatment with AME, EBS, or rottlerin attenuated hyperthermia induced by MA in a dose-related manner (AME 50 mg/kg, EBS 20 mg/kg or rottlerin 20 mg/kg: p<0.01 vs. MA-treated mice). In addition, these protective effects were less pronounced in GPx-1 (−/−) mice (p<0.05) than GPx-1 (+/+) mice (FIG. 6).

### Example 2.5

### Orally-Administered AME, EBS or Rottlerin Attenuated the MA-Induced Behavioral Impairments in the GPx-1 (+/+)- and GPx-1 (−/−)-Mice

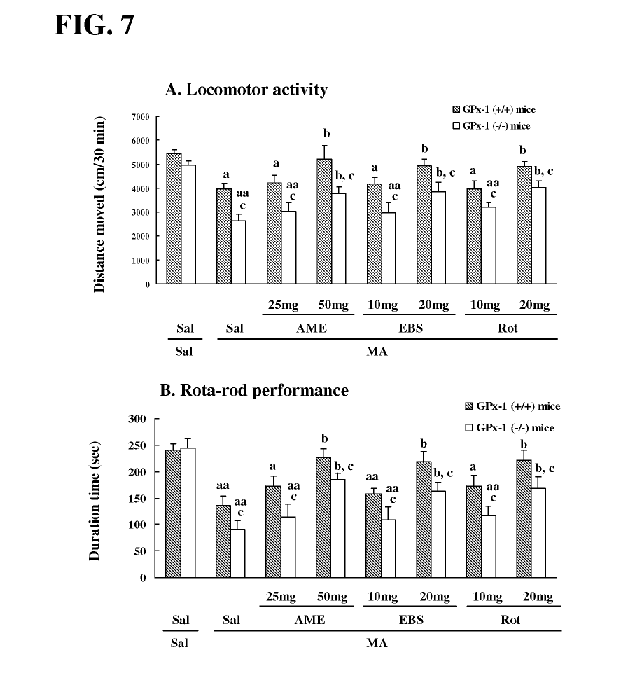

Treatment with MA produced the impairments in the locomotor activity and rota-rod performance [Locomotor activity: p<0.05 vs. saline-treated GPx-1 (+/+), p<0.01 vs. saline-treated GPx-1 (−/−). Rota-rod performance: p<0.05 vs. each strain of saline-treated mice]. These changes were more pronounced in the GPx-1 (−/−) mice [p<0.05 vs. MA-treated GPx-1 (+/+)]. Treatment with AME, EBS or rottlerin inhibited these effects in a dose-dependent manner (Each treatment: p<0.05 vs. MA-treated mice) (FIG. 7).

### Example 2.6

### Orally-Administered AME, EBS or Rottlerin Attenuated the MA-Induced Increases in the PKCδ Expressions in the GPx-1 (+/+)- and GPx-1 (−/−)-Mice

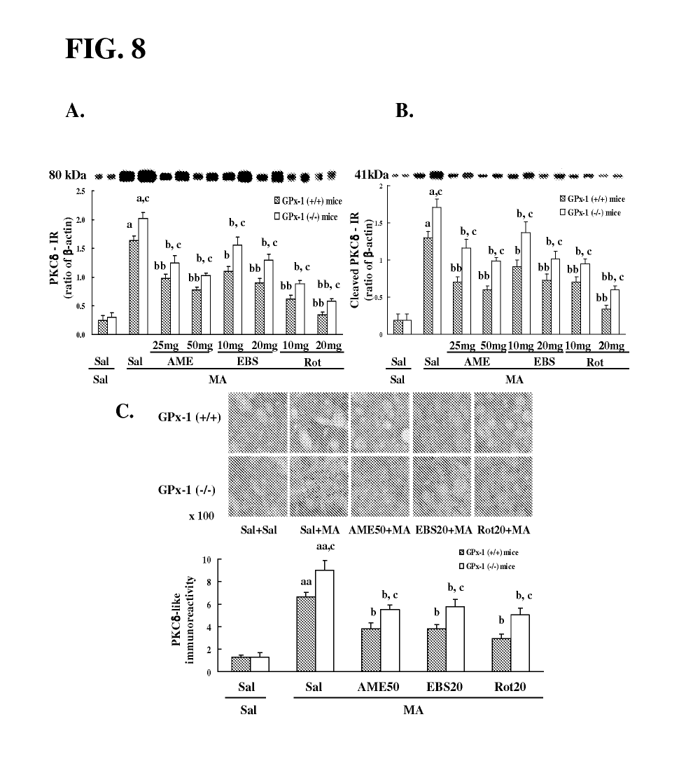

MA administration significantly increased the PKCδ- and cleaved PKCδ-expressions in the striatum of GPx-1 (+/+)- and GPx-1 (−/−)-mice [p<0.01 vs. saline-treated GPx-1 (+/+), p<0.05 vs. saline-treated GPx-1 (−/−)]. This finding was more pronounced in GPx-1 (−/−) mice (p<0.05) than GPx-1 (+/+) mice. Treatment with AME, EBS or rottlerin significantly reduced MA-induced the PKCδ- and cleaved PKCδ-expressions in a dose-related manner [AME 50 mg/kg or EBS 20 mg/kg: p<0.01 vs. MA-treated GPx-1 (+/+), p<0.05 vs. MA-treated GPx-1 (−/−); rottlerin 20 mg/kg: p<0.01 vs. MA-treated mice] (FIGS. 8A and 8B).

### Example 2.7

### Orally-Administered AME, EBS or Rottlerin Attenuated the MA-Induced Changes in PKCδ-like Immunoreactivity (PKCδ-IR) in the GPx-1 (+/+)- and GPx-1 (−/−)-Mice

Representative photomicrographs of PKCδ-immunostained striatal section are shown in FIG. 8C. MA administration caused a significant increase of PKCδ-IR in each strain of mice (p<0.01 vs. each strain of saline-treated mice). These were more pronounced in GPx-1 (−/−) mice (p<0.05) than GPx-1 (+/+) mice. Treatment with AME (50 mg/kg), EBS (20 mg/kg) or rottlerin (20 mg/kg) significantly attenuated MA-induced increase in striatal PKCδ-IR (Each treatment: p<0.05 vs MA-treated mice).

### Example 2.8

### Orally-Administered AME, EBS or Rottlerin Attenuated the MA-Induced Decrease in Glutathione Peroxidase-1-Like Immunoreactivity (GPx-1-IR) in the Striatum of the GPx-1 (+/+) Mice

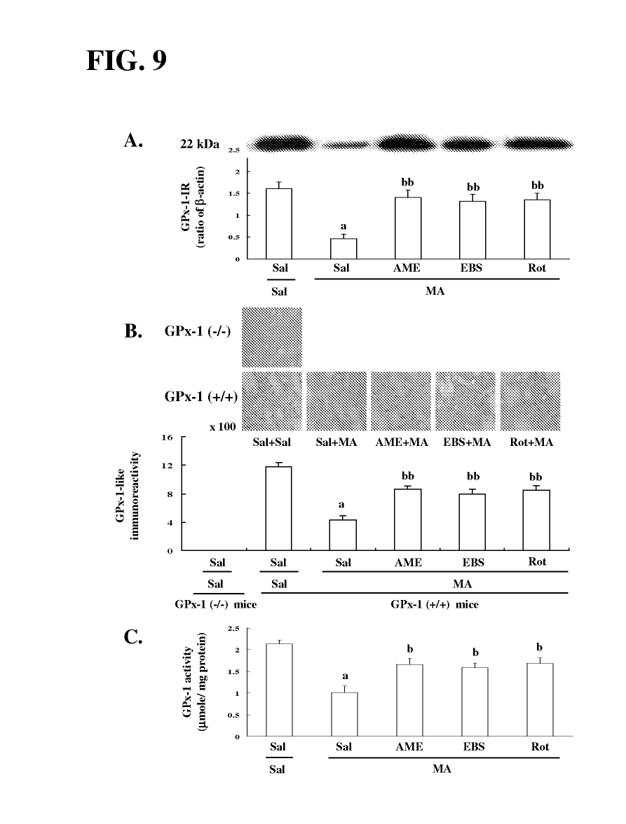

MA-induced decrease (p<0.01 vs. each strain of saline-treated mice) in the GPx-1-IR and GPx-1 activity was observed. Treatment with AME (50 mg/kg), EBS (20 mg/kg) or rottlerin (20 mg/kg) inhibited MA-induced decrease in the GPx-1-IR (p<0.01) and GPx-1 activity (p<0.05) in the striatum of GPx-1 (+/+) mice (FIG. 9).

### Example 2.9

### Orally-Administered AME, EBS or Rottlerin Attenuated the MA-Induced Dopaminergic Impairments in the GPx-1 (+/+)- and GPx-1 (−/−)-Mice

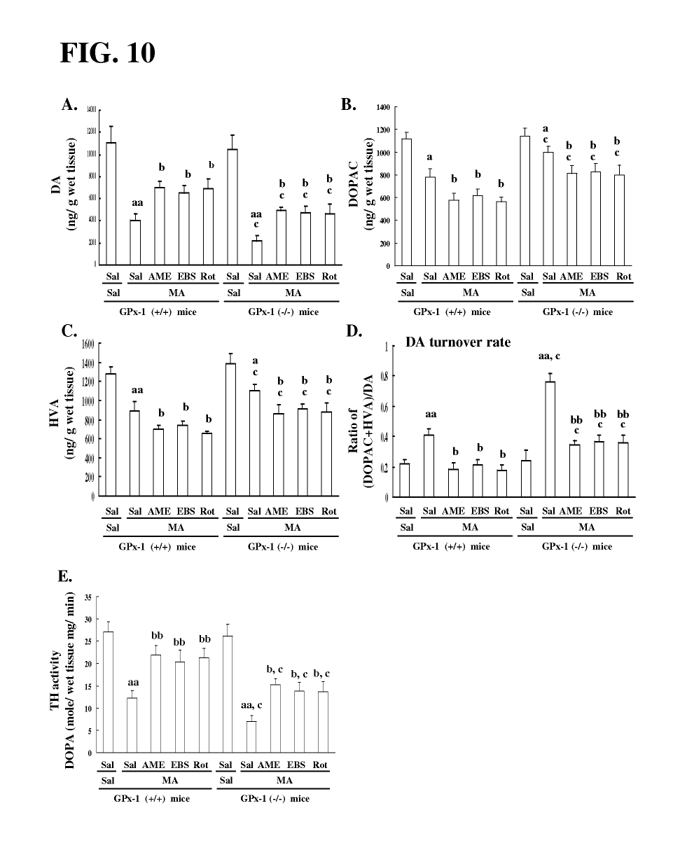

In the absence of MA, dopamine (DA), 3,4-dihydroxyphenylacetic acid (DOPAC), homovanillic acid (HVA) and DA turnover rate did not alter. MA-elicited marked striatal changes in the levels of DA (p<0.01 vs. each strain of saline-treated mice), DOPAC (p<0.05 vs. each strain of saline-treated mice) and HVA [p<0.01 vs. saline-treated GPx-1 (+/+); p<0.05 vs. saline-treated GPx-1 (−/−)] and DA turnover rate (p<0.01 vs. each strain of saline-treated mice) in mice. These impairments were more pronounced in the GPx-1 (−/−) mice than GPx-1 (+/+) mice [DA, DOPAC, HVA or DA turnover rate: p<0.05 vs. MA-treated GPx-1 (+/+)]. Treatment with AME (50 mg/kg), EBS (20 mg/kg) or rottlerin (20 mg/kg) significantly attenuated MA-induced dopaminergic impairment [DA, DOPAC or HVA: p<0.05 vs. MA-treated mice; DA turnover rate: p<0.05 vs. MA-treated GPx-1 (+/+), p<0.01 vs. MA-treated GPx-1 (−/−)]. In addition, tyrosine hydroxylase (TH) activity was decreased after MA treatment in each strain of mice (p<0.01 vs. each strain of saline-treated mice). This decrease was more evident in the GPx-1 (−/−) mice (p<0.05) than GPx-1 (+/+) mice. The decrease in TH activity was attenuated by treatment with AME (50 mg/kg), EBS (20 mg/kg) or rottlerin (20 mg/kg) [Each treatment: p<0.01 vs. MA-treated GPx-1 (+/+). Each treatment: p<0.05 vs. MA-treated GPx-1 (−/−)], and this attenuation was less evidenced in the GPx-1 (−/−) mice (p<0.05) than GPx-1 (+/+) mice (FIG. 10).

### Example 2.10

### Orally-Administered AME, EBS or Rottlerin Attenuated the MA-Induced Reduction in Tyrosine Hydroxylase-Like Immunoreactivity (TH-IR) in the GPx-1 (+/+)- and GPx-1 (−/−)-Mice

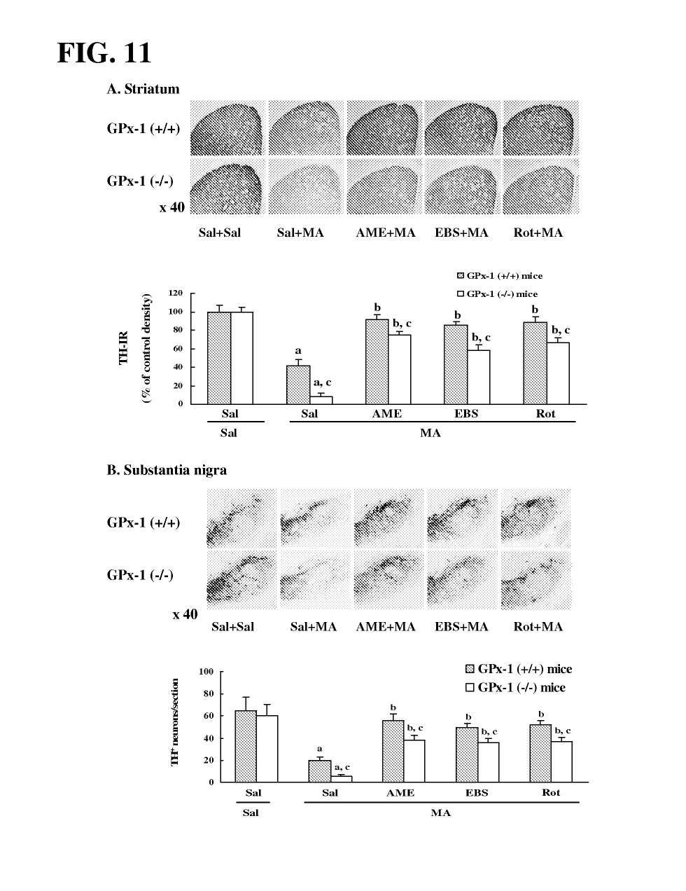

MA administration significantly reduced striatal TH-IR (p<0.01 vs. each strain of saline-treated mice). This reduction was more pronounced in GPx-1 (−/−) mice (p<0.05) than GPx-1 (+/+). Treatment with AME (50 mg/kg), EBS (20 mg/kg) or rottlerin (20 mg/kg) also attenuated this reduction in striatal TH-IR in each strain of mice (p<0.01 vs. each strain of MA-treated mice). However, this attenuation was less evident in GPx-1 (−/−) mice (p<0.05) than GPx-1 (+/+) mice (FIG. 11A). Consistently, above finding is in line with nigral TH-IR [p<0.01 vs. each strain of saline-treated mice, MA-treated GPx-1 (+/+) mice vs. MA-treated GPx-1 (−/−) mice: p<0.01]. Treatment with AME (50 mg/kg), EBS (20 mg/kg) or rottlerin (20 mg/kg) significantly attenuated MA-induced nigral reduction in TH-IR (p<0.01 vs. each strain of MA-treated mice) (FIG. 11B).

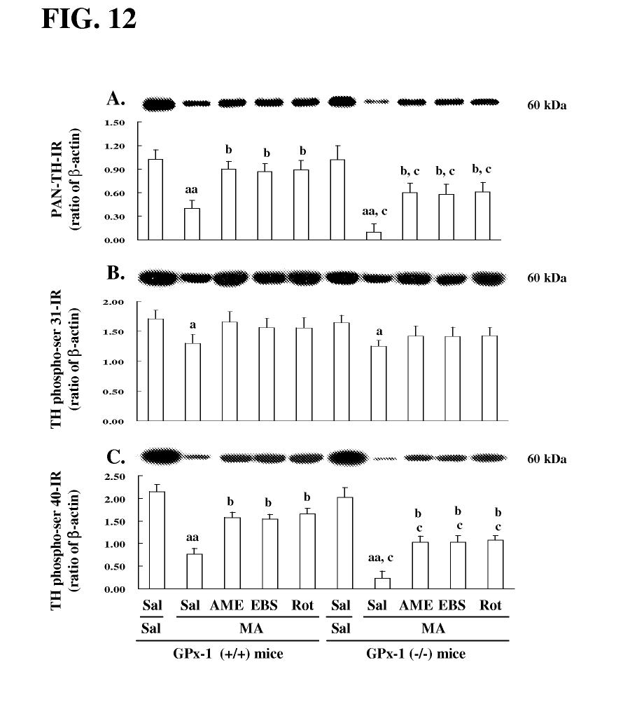

MA-induced reduction in PAN-TH expression was observed (p<0.01 vs. each strain of saline-treated mice). These reductions might be attributable to reduction in phospho-TH-ser 40 (p<0.01 vs. each strain of saline-treated mice), although MA-induced reduction in phospho-TH-ser 31 (p<0.05 vs. each strain of saline-treated mice) was observed. Treatment with AME (50 mg/kg), EBS (20 mg/kg) or rottlerin (20 mg/kg) significantly blocked (p<0.05 for each case) MA-induced decrease in phospho-TH-ser 40, and these effects were less pronounced in GPx-1 (−/−) mice (p<0.05) than GPx-1 (+/+) mice (FIG. 12).

### Example 2.11

### Orally-Administered AME, EBS or Rottlerin Attenuated the MA-Induced Alterations in p53 and Mouse Double Minute (MDM) 2 in the GPx-1 (+/+)- and GPx-1 (−/−)-Mice

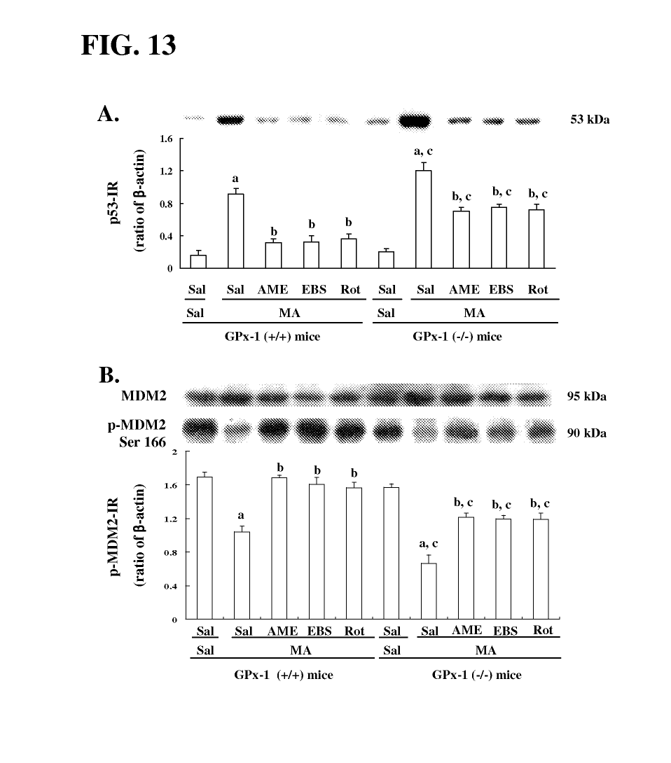

The striatal expression of p53 protein was markedly increased after MA administration in each strain of mice (p<0.01 vs. each strain of saline-treated mice). This change was more pronounced in GPx-1 (−/−) mice (p<0.05) than GPx-1 (+/+) mice (FIG. 13A). Treatment with AME (50 mg/kg), EBS (20 mg/kg) or rottlerin (20 mg/kg) significantly inhibited MA-induced increase in p53 expression (p<0.01 vs. each strain of MA-treated mice).

Although no significant change in the MDM2 expression was observed in the striatum after the final MA injection, phospho-MDM2 expression was significantly decreased (p<0.01 vs. each strain of saline-treated mice). This decrease was more evident in GPx-1 (−/−) mice (p<0.05) than GPx-1 (+/+) mice. AME (50 mg/kg), EBS (20 mg/kg) or rottlerin (20 mg/kg) significantly attenuated (p<0.01) this decrease in each strain of MA-treated mice (FIG. 13B).

### Example 2.12

### Orally-Administered AME, EBS or Rottlerin Attenuated the MA-Induced Altered Expression in Neuroinflammatory Factors in the GPx-1 (+/+)- and GPx-1 (−/−)-Mice

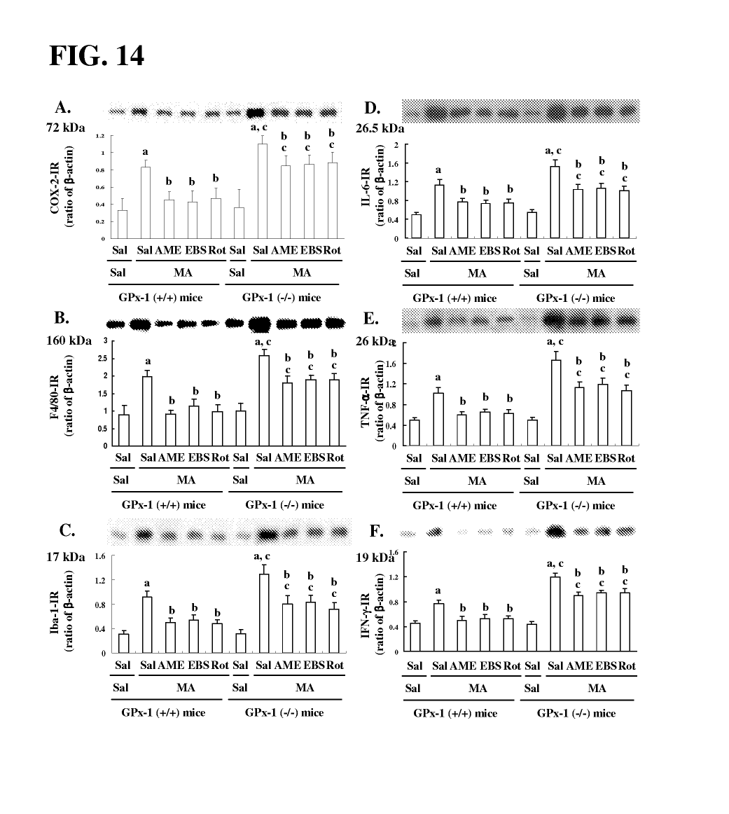

MA-induced increases in the cyclooxygenase (COX)-2-, F4/80 (a marker of microgliosis)-, Iba-1 (a marker of microgliosis)-, interleukin (IL)-6-, tumor necrosis factor (TNF)-α-, and interferon (IFN)-γ-expressions were observed (p<0.01 vs. each strain of saline-treated mice). These expressions of proinflammation factors showed in a similar pattern. These expressions were consistently more evident in GPx-1 (−/−) mice (p<0.05) than GPx-1 (+/+) mice (FIG. 14).

Although there was no difference in pharmacological action among AME, EBS, and rottlerin, each one exerted significantly and consistently attenuating effects (p<0.05) against activation of these proinflammatory factors.

### Example 2.13

### Orally-Administered AME, EBS or Rottlerin Attenuated the MA-Induced Reductions in Neurotrophic Factors in the GPx-1 (+/+)- and GPx-1 (−/−)-Mice

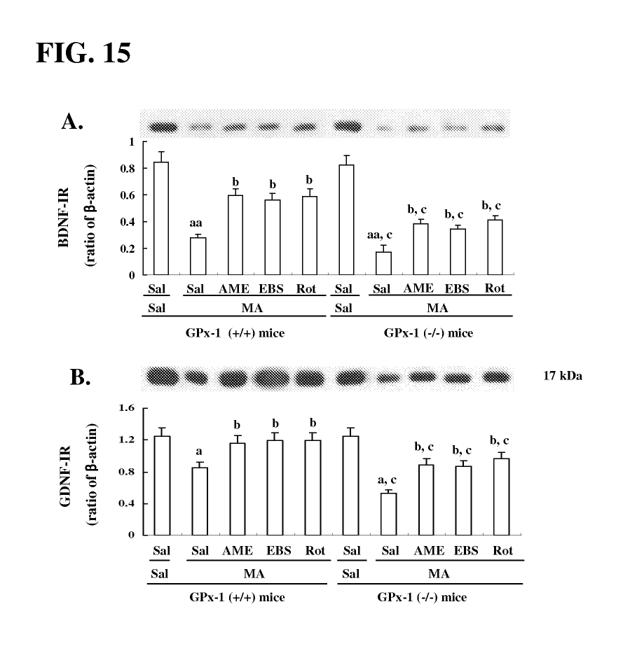

MA-induced significant reductions in brain-derived neurotrophic factor (BDNF) and glial cell line-derived neurotrophic factor (GDNF) expressions were observed (BDNF: p<0.01 vs. each strain of saline-treated mice. GDNF: p<0.05 vs. each strain of saline-treated mice). These reductions were consistently more evident in GPx-1 (−/−) mice (p<0.05) than GPx-1 (+/+) mice. Treatment with AME (50 mg/kg), EBS (20 mg/kg) or rottlerin (20 mg/kg) significantly attenuated these reductions (p<0.05 vs. each strain of MA-treated mice) (FIG. 15).

### Example 2.14

### Orally-Administered AME, EBS or Rottlerin Attenuated MA-Induced Oxidative Stresses in the GPx-1 (+/+)- and GPx-1 (−/−)-Mice

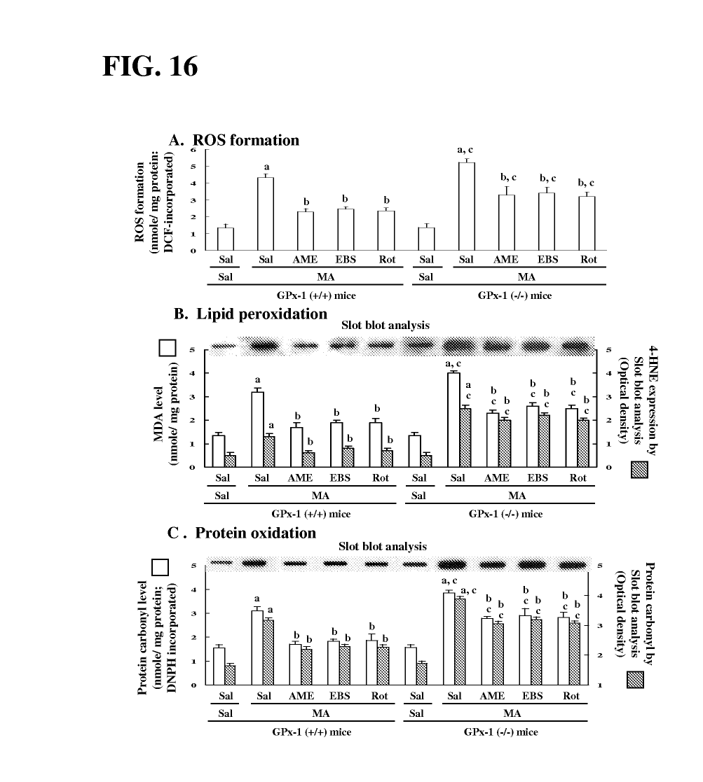

The striatal changes in the oxidative stress markers [as measured by reactive oxygen species (ROS), protein carbonyl oxidation and lipid peroxidation] were evaluated at 3 days after the final of MA administration. The significant increases in ROS, protein oxidation [as measured by biochemical assay and slot blot analysis] and lipid peroxidation [as measured by biochemical formation of MDA and by 4-HNE expression (slot blot analysis)] were observed in MA-treated mice (p<0.01 vs. each strain of saline-treated mice). These changes were consistently more pronounced in GPx-1 (−/−) mice (p<0.05) than GPx-1 (+/+) mice. These increases were significantly attenuated (p<0.05) by the treatment with AME (50 mg/kg), EBS (20 mg/kg) or rottlerin (20 mg/kg) in each strain of mice (FIG. 16).

TABLE 1
Application of protein kinase C (PKC) inhibitors

| | |
|---|---|
| Classification | Dose and route |

| | | |
|---|---|---|
| 1. | Go 6976: PKCα and β inhibitor. | 1.0 μg/2 μl/intra-striatal. |
| | (conventional PKC inhibitor) | 2.0 μg/2 μl/intra-striatal. |
| 2. | Hispidin: PKCβ inhibitor. | 1.5 μg/3 μl/intra-striatal. |
| | (conventional PKC inhibitor) | 3.0 μg/3 μl/intra-striatal. |
| 3. | Rottlerin: PKCδ inhibitor. | 1.5 μg/1.5 μl/intra-striatal. |
| | (novel PKC inhibitor) | 3.0 μg/1.5 μl/intra-striatal. |
| 4. | PKC zeta pseudosubstrate | 1.5 μg/3 μl/intra-striatal. |
| | inhibitor: PKCζ inhibitor. | 3.0 μg/3 μl/intra-striatal. |
| | (atypical PKC inhibitor) | |
| 5. | Chelerythrine chloride: | 0.1 μg/2 μl/intra-striatal. |
| | pan-PKC inhibitor. | 0.2 μg/2 μl/intra-striatal. |

### Example 3

### Materials and Methods

### Example 3.1

### Animal

All mice were treated in strict accordance with the NIH Giude for the Humane Care and Use of Laboratory Animals (NIH Guide for the Care and Use of Laboratory Animals). C57BL/6 mice weighing about 27±3 g were maintained on a 12 h/12 h light/dark cycle and fed ad libitum. They were adapted for 2 weeks to the above conditions before experimentation.

### Example 3.2

### Structure of Drug

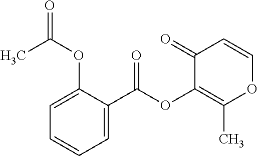

Acetylsalicylic acid maltol ester [3-(methyl-4-pyrinyl)-2-acetyloxybenzoate; AME]

### Example 3.3

### Drug Treatments

AME (25 mg/kg, p.o) administration was started 4 days (twice daily) before the first injection of MPTP and continued for 7 consecutive days (once a day). MPTP was injected (25 mg/kg, i.p) once a day 1 h after AME treatment for consecutive 7 days.

### Example 3.4

### Locomotor Activity

Locomotor activity measured for 30 min 3 days after the last MPTP administration using an automated video-tracking system (Noldus Information Technology, Wagenin, The Netherlands). Eight test boxes (40×40×30 cm high) were operated simultaneously by an IBM computer. Animals were studied individually during locomotion in each test box, where they were adapted for 5 min before starting the experiment. A printout for each session showed the pattern of the ambulatory movements of the test box. The distance traveled in cm by the animals in horizontal locomotor activity was analyzed. Data were collected and analyzed between 09:00 and 17:00 h (Kim et al., 2001).

### Example 3.5

### Rota-Rod Test

The apparatus (Ugo Basile model 7650) consisted of a base platform and a rotating rod with a nonslippery surface. The rod was placed at a height of 15 cm from the base. The rod, 30 cm in length, was divided into 5 equal sections by 6 opaque disks (so that the subjects cannot be distracted by one another). To assess motor performance, the mice were first trained on the apparatus 2 minutes at a rate 4 r.p.m. per 30 s prior to the test. The test was performed 30 minutes after training and an accelerating paradigm was applied at a rate 4 r.p.m. per 30 s, starting from 4 r.p.m. to a maximum speed of 40 r.p.m., then the rotation speed was kept constant at 40 r.p.m. for a maximum of 300 s. The duration for which the animal could maintain balance on the rotating drum was measured as the rotarod latency, with a maximal cut-off time of 300 s.

### Example 3.6

### Immunocytochemistry

Animals were sacrificed at 3 days after final MPTP-treatment. They were anesthetized with 60% urethane and perfused transcardially with 200 ml of 50 mM phosphate buffered saline (PBS), followed by 50 ml of paraformaldehyde in PBS. The brain were fixed at 4° C. for 24 h in the same fixative and then cryoprotected in 30% sucrose. The brains were sectioned on a horizontal sliding microstome into 35 μm transverse free-floating sections. The immunocytochemistry was performed as described previously (Kim et al., 2000a; Kim et al., 2000b). Briefly, prior to incubation with the primary antibodies, sections were preincubated with 0.3% hydrogen peroxide in PBS for 30 min (to block endogenous peroxidase activity), then in PBS containing 0.4% Triton X-100 for 20 min and 1% normal serum for 20 min. After a 48 h incubation with the primary antibody at 4° C., sections were incubated with the secondary biotinylated antisera (1:1000 dilution; Vector, Brulingame, Calif.) for 1 h, washed, and immersed in avidin-biotin-peroxidase complex (ABC Elite kit, Vector) for 1 hr. Sections were always washed three times with PBS between each incubation step. 3,3′-Diaminobenzidine (DAB) was used as the chromogen. The quantitative analyses were performed using a computer-based image analysis system (Optimas version 6.2; Neurolucida Program) (Kim et al., 1999).

### Example 3.7

### HPLC Analysis

At 3 days after last MPTP injection, mice were killed by cervical dislocation. The brains were removed and placed on an ice-cooled plate. Striatum was dissected and immediately frozen on dry ice and stored at −70° C. until extraction. Brain regions obtained from each animal were weighed, ultrasonicated in 10% perchloric acid containing 10 ng/mg of the internal standard dihydroxybenzilamine, and centrifuged at 20,000×g for 10 min. The levels of DA and its metabolites 3,4-dihydroxyphenylacetic acid (DOPAC) and homovanillic acid (HVA) in brain tissue extracts were determined by HPLC coupled with electrochemical detection as described (Kim et al., 1999). Briefly, striatal tissues were sonicated in 0.2 M perchloric acid (20% W/V) containing the internal standard 3,4-dihydroxybenzylamine (10 mg wet tissue/ml). The homogenate was centrifuged and a 20 μl aliquot of the supernatant was injected into the HPLC equipped with a 3 μm C18 column. The mobile phase was comprised of 26 ml of acetonitrile, 21 ml of tetrahydrofuran and 960 ml of 0.15 M monochloroacetic acid (pH 3.0) containing 50 mg/l of EDTA and 200 mg/l of sodium octyl sulfate. The amount of DA, DOPAC and HVA were determined by comparison of peak height ratio of tissue sample with standards, and were expressed in nanograms per gram of wet weight of tissue.

### Example 3.8

### Statistics

Statistical significance was analyzed by one-way ANOVA. Post-hoc Fischer's PLSD test was followed for the comparison among groups. P values<0.05 were deemed to be statistically significant.

### Example 4

### Results

### Example 4.1

### Effects of AME on MPTP-Induced Behavioral Changes in Mice

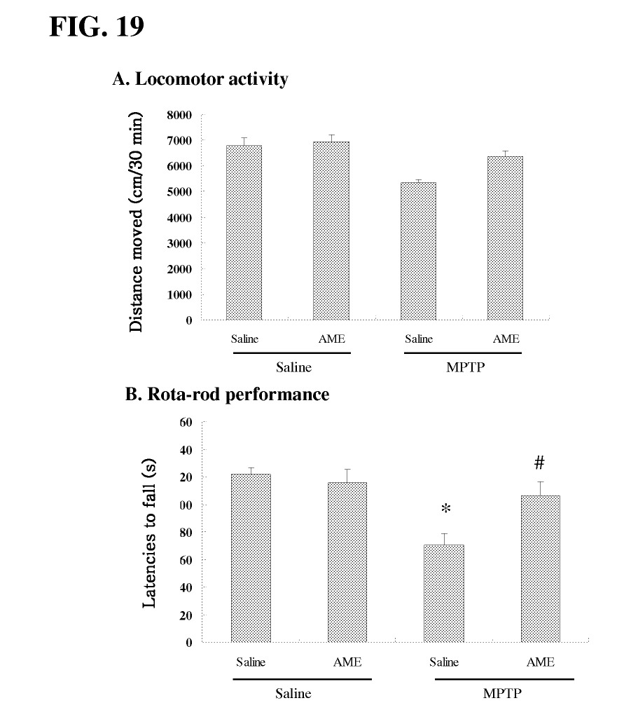

Effects of AME on the changes in the locomotor activity and rota-rod performance were shown in FIGS. 19A and 19B. MPTP-treated mice showed a significant hypolocomotor activity (p<0.05 vs. saline), which was significant attenuated by AME treatment (p<0.05 vs. MPTP alone) (FIG. 19A).

Rota-rod performance was also impaired in the MPTP-treated mice (p<0.05 vs. saline). MPTP-induced impairment in the rota-rod performance was significantly attenuated by AME treatment (p<0.05 vs. MPTP alone) (FIG. 19B).

### Example 4.2

### Effects of AME on MPTP-Induced Dopamine Level and its Metabolites

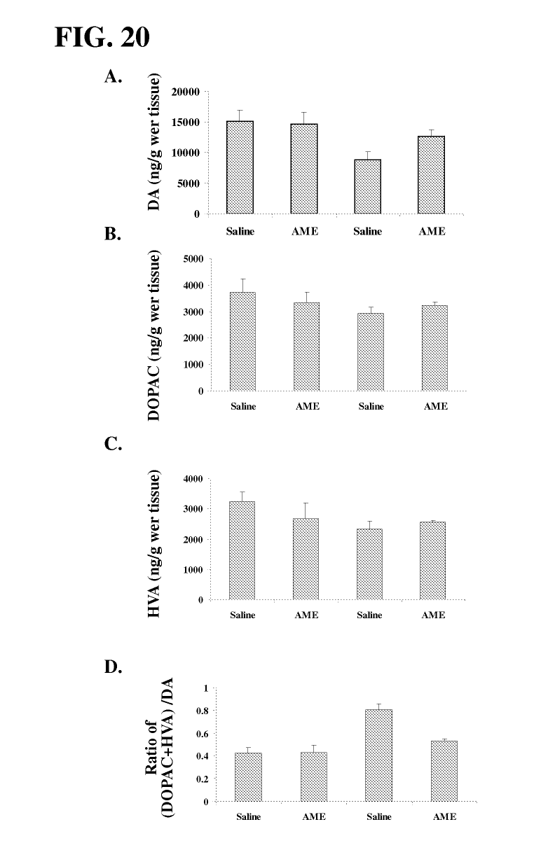

After the final MPTP injection, the levels of dopamine and its metabolites in the striatum were measured (FIG. 20). Administration of MPTP led to a marked reduction in striatal dopamine (DA) (p<0.01 vs. saline), 3,4-Dihydroxyphenylacetic acid (DOPAC) (p<0.01 vs. saline) and homovanillic acid (HVA) (p<0.01 vs. saline). These reductions were significant attenuated, respectively by AME treatment (DA: p<0.01 vs. MPTP alone; DOPAC: p<0.05 vs. MPTP alone; HVA: p<0.01 vs. MPTP alone).

DA turnover rate was significantly increased in MPTP-treated mice (p<0.01 vs. saline). This DA turnover rate with significant attenuated (p<0.01 vs. MPTP alone) by AME.

### Example 4.3

### Effects of AME on MPTP-Induced Reductions in Tyrosine Hydroxylase-Like Immunoreactivity (TH-IR)

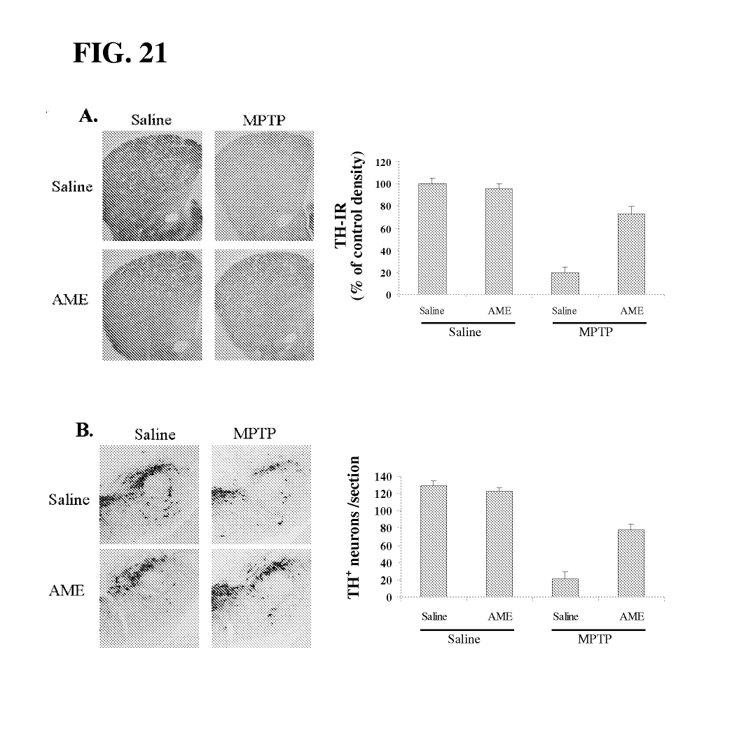

FIG. 21 showed nigrostriatal TH-IR. MPTP-induced decreases in TH-IR in both striatum (A) and substantia nigra (B) were observed (striatum: p<0.01 vs. saline; SN: p<0.01 vs. saline). AME treatment significantly attenuated these decreases in TH-IR (striatum: p<0.01 vs. MPTP alone; SN: p<0.01 vs. MPTP alone).

### REFERENCES

Amstad, P., Peskin, A., Shah, G., Mirault, M. E., Moret, R., Zbinden, I., and Cerutti, P. The balance between Cu,Zn-superoxide dismutase and catalase affects the sensitivity of mouse epidermal cells to oxidative stress. Biochemistry, 1991; 30: 9305-9313.
Basu, A., Woolard, M. D., Johnson, C. L. Involvement of protein kinase C-delta in DNA damage-induced apoptosis. Cell Death Differ. 2001; 8(9): 899-908.
Benedetti, A., Comporti, M., Esterbauer, H. Identification of 4-hydroxynonenal as a cytotoxic product originating from the peroxidation of liver microsomal lipids. Biochim. Biophys. Acta. 1980; 620(2): 281-96.
Biswas, S. C., Ryu, E., Park, C., Malagelada, C., Greene, L. A. Puma and p53 play required roles in death evoked in a cellular model of Parkinson disease. Neurochem. Res. 2005; 30: 839-845.
Bourre, L., Thibaut, S., Briffaud, A., Rousset, N., Eleouet, S., Lajat, Y., and Patrice, T. Indirect detection of photosensitizer ex vivo. J. Photochem. Photobiol B. 2002; 67(1): 23-31.
Bowenkamp, K. E., Hoffman, A. F., Gerhardt, G. A., Henry, M. A., Biddle, P. T., Hoffer, B. J., Granholm, A. C. Glial cell line-derived neurotrophic factor supports survival of injured midbrain dopaminergic neurons. J. Comp. Neurol. 1995; 355(4): 479-489.
Browman, K. E., Kantor, L., Richardson, S., Badiani, A., Robinson, T. E., Gnegy, M. E. Injection of the protein kinase C inhibitor Ro31-8220 into the nucleus accumbens attenuates the acute response to amphetamine: tissue and behavioral studies. Brain Res. 1998; 814: 112-119.
Cadet, J. L., and Brannock, C. Free radicals and the pathobiology of brain dopamine systems, Neurochem. Int. 1998; 32: 117-131.
Cadet, J. L., Sheng, P., Ali, S., Rothman, R., Carlson, E., Epstein, C. Attenuation of methamphetamine-induced neurotoxicity in copper/zinc superoxide dismutase transgenic mice, J. Neurochem. 1994; 62: 380-383.
Castano, A., Cano, J., Machado, A. Low selenium diet affects monoamine turnover differentially in substantia nigra and striatum, J. Neurochem. 1993; 61: 1302-1307.
Cubells, J. F., Rayport, S., Rajendran, G., and Sulzer, D. Methamphetamine neurotoxicity involves vacuolation of endocytic organelles and dopamine-dependent intracellular oxidative stress. J. Neurosci. 1994; 14: 2260-2271.
Davidson, C., Gow, A. J., Lee, T. H., Ellinwood, E. H. Methamphetamine neurotoxicity: necrotic and apoptotic mechanisms and relevance to human abuse and treatment. Brain Res. Brain Res. Rev. 2001; 36(1): 1-22.
Davies, S. P., Reddy, H., Caivano, M., Cohen, P. Specificity and mechanism of action of some commonly used protein kinase inhibitors. Biochem J. 2000; 351(Pt 1): 95-105.
De Vito, M. J. and Wagner, G. C., Methamphetamine-induced neuronal damage: a possible role for free radicals, Neuropharmacology, 1989; 28: 1145-1150.
Deng, X., Wang, Y., Chou, J., Cadet, J. L. Methamphetamine causes widespread apoptosis in the mouse brain: evidence from using an improved TUNEL, histochemical method. Mol. Brain. Res. 2001; 93: 64-69.
Duan, C. L., Su, Y., Zhao, C. L., Lu, L. L., Xu, Q. Y., and Yang, H. The assay of activities and function of TH, AADC, and GCH1 and their potential use in ex-vivo gene therapy of PD. Brain Res. Protoc. 2005; 16(1-3): 37-43.
Duan, W., Zhu, X., Ladenheim, B., Yu, Q. S., Guo, Z., Oyler, J., Cutler, R. G., Cadet, J. L., Greig, N. H., Mattson, M. P. p53 inhibitors preserve dopamine neurons and motor function in experimental parkinsonism. Ann. Neurol. 2002; 52(5): 597-606.
Fiebich, B. L., Butcher, R. D., Gebicker-Haerter, P. J. Protein kinase C-mediated regulation of inducible nitric oxide synthase expression in cultured microglial cells. J. Neuroimmunol. 1998; 92: 170-178.
Fumagalli, F., Gainetdinov, R. R., Wang, Y. M., Valenzano, K. J., Miller, G. W., Caron, M. G. Increased methamphetamine neurotoxicity in heterozygous vesicular monoamine transporter 2 knock-out mice, J. Neurosci. 1999; 19: 2424-2431.
Ghanooni, R., Decaestecker, C., Simon, P., Gabius, H. J., Hassid, S., Choufani, G. Characterization of patterns of expression of protein kinase C-α, -δ, -η, -γ, and -ξ and their correlations to p53, galectin-3, the retinoic acid receptor-13 and the macrophage migration inhibitory factor (MIF) in human cholesteatomas. Hear. Res. 2006; 214: 7-16.
Gemma, C., Stellwagen, H., Fister, M., Coultrap, S. J., Mesches, M. H., Browning, M. D., and Bickford, P. C. Rosiglitazone improves contextual fear conditioning in aged rats. Neuroreport. 2004; 15(14): 2255-9.
Giovanni, A., Liang, L. P., Hastings, T. G., Zigmond, M. J. Estimating hydroxyl radical content in rat brain using systemic and intraventricular salicylate: impact of methamphetamine, J. Neurochem. 1995; 64: 1819-1822.
Gluck, M. R., Moy, L. Y., Jayatilleke, E., Hogan, K. A., Mizuno, L., and Sonsalla, P. K. Parallel increases in lipid and protein oxidative markers in several mouse brain regions after methamphetamine treatment. J. Neurochem. 2001; 79:152-160.
Guilarte, T. R. Is methamphetamine abuse a risk factor in parkinsonism?. Neurotoxicology 2001; 22(6): 725-731.
Halliwell, B. Reactive ooxygen species and the central nervous system, J. Neurochem. 1992; 59: 1609-1623.
Han, B. H., Suh, D. Y., Tang, H. O., Park, Y. H., Kim, Y. C. Synthesis and antiplatelet effects of the new antithrombotic agent aspalatone with low ulcerogenicity. Arzneim-Forschl Drug Res. 1994; 44: 1122-1126.
Haupt, Y., Maya, R., Kazaz, A., Oren, M. MDM2 promotes the rapid degradation of p53. Nature 1997; 387(6630): 296-299.
Heikkila, R. E., Hess, A., Duvoisin, R. C. Dopaminergic neurotoxicity of 1-methyl-4-phenyl-1,2,3,6-tetrahydropyridine in mice. Science. 1984; 224: 1451-53
Horger, B. A., Nishimura, M. C., Armanini, M. P., Wang, L. C., Poulsen, K. T., Rosenblad, C., Kirik, D., Moffat, B., Simmons, L., Johnson, E. Jr., Milbrandt, J., Rosenthal, A., Bjorklund, A., Vandlen, R. A., Hynes, M. A., Phillips, H. S. Neurturin exerts potent actions on survival and function of midbrain dopaminergic neurons. J. Neurosci. 1998; 18(13): 4929-4937.
Huang, K., Lauridsen, E., Clausen, J. The uptake of Na-selenite in rat brain, Biol. Trace Elem. Res. 1994; 46: 91-102.
Hyman, C., Hofer, M., Barde, Y. A., Juhasz, M., Yancopoulos, G. D., Squinto, S. P., Lindsay, R. M. BDNF is a neurotrophic factor for dopaminergic neurons of the substantia nigra. Nature. 1991; 350(6315): 230-232.
Jakowec, M. W., Nixon, K., Hogg, E., McNeill, T., Petzinger, G. M. Tyrosine hydroxylase and dopamine transporter expression following 1-methyl-4-phenyl-1,2,3,6-tetrahydropyridine-induced neurodegeneration of the mouse nigrostriatal pathway. J Neurosci Res. 2004; 76: 539-50.
Jareno, E. J., Bosch-Morell, F., Fernandez-Delgado, R., Donat, J., Romero, F. J. Serum malondialdehyde in HIV seropositive children. Free Radic. Biol. Med. 1998; 24: 503-506.
Jayanthi, S., Ladenheim, B., and Cadet, J. L. Methamphetamine-induced changes in antioxidant enzymes and lipid peroxidation in copper/zinc-superoxide dismutase transgenic mice. Ann. N.Y. Acad. Sci. 1998; 844: 92-102.
Jimenez-Jimenez, F. J., Molina, J. A., Arrieta, F. J., Aguilar, M. V., Cabrera-Baldivida, F., Vazquez, A., Jorge-Santamaria, A., Seijas, V., Fernandez-Calle, P., Marinez-Para, M. C. Decreased serum selenium concentrations in patients with Parkinson's disease. Eur. J. Neurol. 1995; 2: 111-114.
Johnson, L. A., Guptaroy, B., Lund, D., Shamban, S., and Gnegy, M. E. Regulation of amphetamine-stimulated dopamine efflux by protein kinase C β. J. Biol. Chem. 2005; 280: 10914-10919.
Kantor, L., Gnegy, M. E. Protein kinase C inhibitors block amphetamine-mediated dopamine release in rat striatal slices, J. Pharmacol. Exp. Ther. 1998; 284: 594-598.
Kaul, S., Anantharam, V., Yang, Y., Choi, C. J., Kanthasamy, A., Kanthasamy, A. G. Tyrosine phosphorylation regulates the proteolytic activation of protein kinase Cδ in dopaminergic neuronal cells. J. Biol. Chem. 2005; 280: 28721-28730.
Kim, C, Nam, S W, Choi, D Y, Choi, J H, Park, E S, Joo, W K, Kim, H C. A new antithrombotic agent, aspalatone, attenuated cardiotoxicity induced by doxorubicin in the mouse; possible involvement of antioxidant mechanism. Life Sci., 1997a; 60(45): PL75-82.
Kim, C., Koo, C. H., Choi, D. Y., Cho, Y. J., Choi, J. H., Im, D. H., Jhoo, W. K., and Kim, H. C. The effect of aspalatone, a new antithrombotic agent, on the specific activity of antioxidant enzyme in the rat blood. Arch. Pharm. Res. 1996; 19: 348-352.
Kim, H. C., Bing, G., Jhoo, W. K., Suh, J. H., Shin, E. J., Kato, K., Ko, K. H. An immunocytochemical study of mitochondrial manganese superoxide dismutase in the rat hippocampus after kainate administration. Neurosci. Lett. 2000a; 281: 65-68.
Kim, H. C., Bing, G., Jhoo, W. K., Kim, W. K., Shin, E. J., Park, E. S., Choi, Y. S., Lee, D. W., Shin, C. Y., Ryu, J. R., Ko, K. H. Oxidative damage causes formation of lipofuscin-like substances in the hippocampus of the senescence-accelerated mouse after kainate treatment. Behay. Brain Res. 2002; 131: 211-220.
Kim, H. C., Bing, G., Shin, E. J., Jhoo, H. S., Cheon, M. A., Lee, S. H., Choi, K. H., Kim, J. I., and Jhoo, W. K. Dextromethorphan affects cocaine-mediated behavioral pattern in parallel with a long-lasting Fos-related antigen-immunoreactivity. Life Sci. 2001; 69(6): 615-624.
Kim, H. C., Jhoo, W. K., Bing, G., Shin, E. J., Wie, M. B., Kim, W. K., and Kato, K. H. Phenidone prevents kainite-induced neurotoxicity via antioxidant mechanisms. Brain Res. 2000b; 874: 15-23.
Kim, H. C., Jhoo, W. K., Choi, D. Y., Im, D. H., Shin, E. J., Suh, J. H., Floyd, R. A., Bing, G. Protection of methamphetamine nigrostriatal toxicity by dietary selenium, Brain Res. 1999; 851: 76-86.
Kim, H. C., Jhoo, W. K., Shin, E. J., Bing, G. Selenium deficiency potentiates methamphetamine-induced nigral neuronal loss; comparison with MPTP model, Brain Res. 2000c; 862: 247-252.
Kim, H C, Choi, D Y, Jhoo, W K, Lee, D W, Koo, C H, Kim, C. Aspalatone, a new antiplatelet agent, attenuates the neurotoxicity induced by kainic acid in the rat. Life Sci. 1997b; 61(24): PL373-81.
Kim, H. C., Yamada, K., Nitta, A., Olariu, A., Tran, M. H., Mizuno, M., Nakajima, A., Nagai, T., Kamei, H., Jhoo, W. K., Im, D. H., Shin, E. J., Hjelle, O. P., Ottersen, O. P., Park, S. C., Kato, K., Mirault, M. E., Nabeshima, T. Immunocytochemical evidence that amyloid β (1-42) impairs endogenous antioxidant systems in vivo. Neuroscience 2003; 119: 399-419.
Kim, S., Westphalen, R., Callahan, B., Hatzidimitriou, G., Yuan, J., Ricaurte, G. A Toward development of an in vitro model of methamphetamine-induced dopamine nerve terminal toxicity. J. Pharmacol. Exp. Ther. 2000; 293: 625-633.
Kita, T., Wagner, G. C., Nakashima, T. Current research on methamphetamine-induced neurotoxicity: animal models of monoamine disruption. J. Pharmacol. Sci. 2003; 92(3): 178-195.
Kreutzberg, G. W. Microglia: a sensor for pathological events in the CNS. Trends Neurosci. 1996; 19: 312-318.
Kubbutat, M. H., Jones, S. N., Vousden, K. H. Regulation of p53 stability by Mdm2. Nature 1997; 387(6630): 299-303.
Lapchak, P. A., Jiao, S., Miller, P. J., Williams, L. R., Cummins, V., Inouye, G., Matheson, C. R., Yan, Q. Pharmacological characterization of glial cell line-derived neurotrophic factor (GDNF): implications for GDNF as a therapeutic molecule for treating neurodegenerative diseases. Cell Tissue Res. 1996; 286: 179-189.
Lapchak, P. A., Zivin, J. A. Ebselen, a seleno-organic antioxidant, is neuroprotective after embolic strokes in rabbits: synergism with low-dose tissue plasminogen activator. Stroke 2003; 34(8): 2013-2018.
LaVoie, M. J., and Hastings, T. G. Dopamine quinine formation and protein modification associated with the striatal neurotoxicity of methamphetamine: evidence against a role for extracellular dopamine, J. Neurosci. 1999; 19: 1484-1491.
LaVoie, M. J., Card, J. P., and Hastings, T. G. Microglial activation precedes dopamine terminal pathology in methamphetamine-induced neurotoxicity. Exp. Neurol. 2004; 187: 47-57.
Lewin, G. R., Barde, Y. A. Physiology of the neurotrophins. Annu. Rev. Neurosci. 1996; 19: 289-317.
Li, R., Peng, N., Li, X-P., Le, W-D. (−)-Epigallocatechin gallate regulates dopamine transportes internalization via protein kinase C-dependent pathway. Brain Res. 2006; 1097(1): 85-89.
Martin-Iverson, M. T., Todd, K. G., Altar, C. A. Brain-derived neurotrophic factor and neurotrophin-3 activate striatal dopamine and serotonin metabolism and related behaviors: interactions with amphetamine. J. Neurosci. 1994; 14(3Pt1): 1262-1270.
McMillian, M. K., Vainio, P. J., Tuominen, R. K. Role of protein kinase C in microglia-induced neurotoxicity in mesencephalic cultures. J. Neuropathol. Exp. Neurol. 1997; 56(3): 301-307.
Miller, R. L., Sun, G. Y., Sun, A. Y. Cytotoxicity of paraquat in microglial cells: Involvement of PKCdelta- and ERK1/2-dependent NADPH oxidase. Brain Res. 2007; 1167: 129-139.
Nair, V. D. Activation of p53 signaling initiates apoptotic death in a cellular model of Parkinson's disease. Appotosis. 2006; 11(6): 955-966.
Olanow, C. W., Obeso, J. A., Stocchi, F. Continuous dopamine-receptor treatment of Parkinson's disease: scientific rationale and clinical implications. Lancet Neurol. 2006; 5: 677-87.
Pierce, R. C., Pierce-Bancroft, A. F., Prasad, B. M. Neurotrophin-3 contributes to the initiation of behavioral sensitization to cocaine by activating the Ras/Mitogen-activated protein kinase signal transduction cascade. J. Neurosci. 1999; 19: 8685-8695.
Przedborski, S., Kostic, V., Jackson-Lewis, V., Naini, A. B., Simonetti, S., Fahn, S., Carson, E., Epstein, C. J., Cadet, J. L. Transgenic mice with increased Cu/Zn-superoxide dismutase activity are resist to N-methyl-4-phenyl-1,2,3,6-tetrahydropyridine-induced neurotoxicity, J. Neurosci. 1992; 12: 1658-1667.
Pubill, D., Chipana, C., Camins, A., Pallas, M., Camarasa, J., Escubedo, E. Free radical production induced by methamphetamine in rat striatal synaptosomes. Toxicol. Appl. Pharmacol. 2005; 204: 57-68.
Rotruck, J. T., Pope, A. L., Ganther, H. E., Swanson, A. B., Hafeman, D., Hoekstra, W. G. Selenium: biochemical role as a component of glutathione peroxidase, Science 1973; 179: 588-590.
Sandoval, V., Riddle, E. L., Ugarte, Y. V., Hanson, G. R., Fleckenstein, A. E. Methamphetamine-induced rapid and reversible changes in dopamine transportes function: an in vitro model. J. Neurosci. 2001; 21: 1413-1419.
Schewe, T. Molecular actions of ebselen-an anti-inflammatory antioxidant. Gen Pharmacol. 1995; 26(6): 1153-1169.
Shen, L., Figurov, A., Lu, B. Recent progress in studies of neurotrophic factors and their clinical implications. J. Mol. Med. 1997; 75: 637-644.
Shin, E. J., Ko, K. H., Kim, W. K., Chae, J. S., Yen, T. P. H., Kim, H. J., Wie, M. B., and Kim, H. C. Role of glutathione peroxidase in the ontogeny of hippocampal oxidative stress and kainite seizure sensitivity in the genetically epilepsy-prone rats. Neurochemistry International 2008; 52: 1134-1147.
Sies, H. Ebselen, a selenoorganic compound as glutathione peroxidase mimic. Free Radic Biol. Med. 1993; 14(3): 313-323.
Sies, H., Arteel, G. E. Interaction of peroxynitrite with selenoproteins and glutathione peroxidase mimic. Free Radic. Biol. Med. 2000; 28(10): 1451-1455.
Sonsalla, P. K., Jochnowitz, N. D., Zeevalk, G. D., Oostveen, J. A., Hall, E. D. Treatment of mice with methamphetamine produces cell loss in the substantia nigra. Brain Res. 1996; 738(1): 172-175.
Spina, M. B., Squinto, S. P., Miller, J., Lindsay, R. M., Hyman, C. Brain-derived neurotrophic factor protects dopamine neurons against 6-hydroxydopamine and N-methyl-4-phenylpyridinium ion toxicity: involvement of the glutathione system. J. Neurochem. 1992; 59(1): 99-106.
Stollg, G., Jander, S. The role of microglia and macrophages in the pathophysiology of the CNS. Prog. Neurobiol. 1999; 58: 233-247
Streit, W. J., Walter, S. A., Pennell, N. A. Reactive microgliosis. Prog. Neurobiol. 1999; 57: 563-581.
Taylor, J. M., Ali, U., Iannello, R. C., Hertzog, P., Crack, P. J. Diminished Akt phosphorylation in neurons lacking glutathione peroxidase-1 (GPx1) leads to increased susceptibility to oxidative stress-induced cell death. J. Neurochem. 2005; 92: 283-293.
Teixeira, H. D., and Meneghini, R. Chinese hamster fibroblasts overexpressing CuZn-superoxide dismutase undergo a global reduction in antioxidants and increase sensitivity of DNA to oxidative damage. Biochem. J., 1995; 315: 821-825.
Thomas, D. M., Francescutti-Verbeem, D.M., Liu, X., and Kuhn, D. M. Identification of differentially regulated transcripts in mouse striatum following methamphetamine treatment—an oligonucleotide microarray approach. J. Neurochem. 2004; 88: 380-393.
Thomas, D. M., Walker, P. D., Benjamins, J. A., Geddes, T. J., Kuhn, D. M. Methamphetamine neurotoxicity in dopamine nerve endings of the striatum is associated with microglial activation. J. Pharmacol. Exp. Ther. 2005; 311: 1-7.
Tipton, K. F., Singer, T. P. Advances in our understanding of the mechanismsof the neurotoxicity of MPTP and related compounds. J. Neurochem. 1993; 61: 1191-206
Tomac, A., Lindqvist, E., Lin, L. F., Ogren, S. O., Young, D., Hoffer, B. J., Olson, L. Protection and repair of the nigrostriatal dopaminergic system by GDNF in vivo. Nature. 1995; 373(6512): 289-290.
Tsao, L. I., Ladenheim, B., Andrews, A. M., Chiueh, C. C., Cadet, J. L., Su, T. P. Delta opioid peptide [D-Ala2,D-Leu5]enkephalin blocks the long-term loss of dopamine transporters induced by multiple administrations of methamphetamine: involvement of opioid receptors and reactive oxygen species. J. Pharmacol. Exp. Ther. 1998; 287: 322-331.
Wagner, G. C., Carelli, R. M. and Jarvis, M. F., Pretreatment with ascorbic acid attenuates the neurotoxic effects of methamphetamine in rats. Res. Commun. Chem. Pathol. Pharmacol. 1985; 47: 221-228.
Yamaguchi, T., Sano, K., Takakura, K., Saito, I., Shinohara, Y., Asano, T., Yasuhara, H. Ebselen in acute ischemic stroke: a placebo-controlled, double-blind clinical trial. Ebselen Study Group. Stroke. 1998; 29(1): 12-17.
Yamamoto, B. K., and Zhu, W. The effects of methamphetamine on the production of free radicals and oxidative stress. J. Pharmacol. Exp. Ther. 1998; 287(1):107-114.
Ye-Shih, H., Jean-Luc, M., Roderick, T. B., Jin, C., Mary, G., Masayoshi, S., and Colin, D. F. Mice deficient in cellular glutathione peroxidase develop normally and show no increased sensitivity to hyperoxia. J. Biol. Chem. 1997; 272: 16644-16651.
Yokoyama H, Tsuchihashi N, Kasai N, Matsue T, Uchida I, Mori N, Ohya-Nishiguchi H, Kamada H. Hydrogen peroxide augmentation in a rat striatum after metamphetamine injection as monitored in vivo by a Pt-disk microelectrode. Biosens Bioelectron, 1997; 12(9-19):1037-1041.
Zhang, C., Walker, L. M., Hinson, J. A., and Mayeux, P. R. (2000). Oxidant stress in rat liver after lipopolysaccharide administration: Effect of inducible nitric oxide synthase inhibition. J. Pharmacol. Exp. Ther. 2000; 293: 968-972.
Zhang, D., Anantharam, V., Kanthasamy, A., Kanthasamy, A. G. Neuroprotective effect of protein kinase Cδ inhibitor rottlerin in cell culture and animal models of Parkinson's disease. J. Pharmacol. Exp. Ther. 2007; 322(3): 913-922.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

1-9. (canceled)

10. A method for treating Parkinson's disease, comprising:
administering to a patient in need of such treatment a composition comprising acetylsalicylic acid maltol ester or a pharmaceutically acceptable salt thereof and a pharmaceutically acceptable carrier or excipient,
wherein the acetylsalicylic acid maltol ester or a pharmaceutically acceptable salt thereof is administered in an amount that is effective to inhibit protein kinase C δ (PKCδ).

11. The method of claim 10, wherein the composition is administered in an amount that is effective to increase a dopamine level of the patient.

12. The method of claim 10, wherein the composition is administered in an amount that is effective to enhance locomotor activity of the patient.

13. The method of claim 10, wherein the composition is provided in a sustained release dosage form.

14. The method of claim 10, wherein the composition is delivered in a capsule.

15. The method of claim 10, wherein the composition comprises about 0.25 to about 250 mg acetylsalicylic acid maltol ester.

16. The method of claim 10, wherein the composition further comprises a neuroprotective agent.
